(19) United States
(12) Patent Application Publication
(10) Pub. No.: US 2019/0172564 A1
(43) Pub. Date: Jun. 6, 2019

(54) EARLY COST PREDICTION AND RISK IDENTIFICATION

(71) Applicant: International Business Machines Corporation, Armonk, NY (US)

(72) Inventors: Rachita Chandra, Cambridge, MA (US); Vijay S. Iyengar, Cortlandt Manor, NY (US); Dmitriy A. Katz, Ossining, NY (US); Karthikeyan Natesan Ramamurthy, Culver City, CA (US); Emily A. Ray, Hastings on Hudson, NY (US); Moninder Singh, Farmington, CT (US); Dennis Wei, White Plains, NY (US); Gigi Y. C. Yuen-Reed, Tampa, FL (US); Kevin N. Tran, Boston, MA (US)

(21) Appl. No.: 15/831,426

(22) Filed: Dec. 5, 2017

(51) Int. Cl. G16H 10/60; G16H 50/30; G06N 99/00; G06F 17/30; G06N 5/02

(52) U.S. Cl. CPC G16H 10/60; G16H 50/30; G16H 15/00; G06F 17/3043; G06N 5/022; G06N 99/005

(57) ABSTRACT

A system may predict costs for a set of members by building and using a predictive pipeline. The pipeline may be built using a set of historical data for training members. A set of member-level features can be identified by performing empirical testing on the set of historical data. The trained configurable predictive pipeline can generate a set of predictive data for each member, using historical test data for a set of testing members. The system can then generate a predictive report for each set of predictive data.

### BACKGROUND

The present disclosure relates to computer systems, and more specifically, to question answering (QA) systems.

Recent research has been directed to developing QA systems designed to receive input questions, analyze them, and return applicable answers. These systems may rely on natural language processing, automated reasoning, machine learning, and other advanced techniques. Using these techniques, QA systems may provide mechanisms for searching large sources of content and analyzing the content with regard to a given input question in order to determine an answer to the question. In some QA systems, this may take the form of hypothesis generation, scoring, and ranking to determine a final set of one or more output answers. An example of a QA system is IBM's WATSON system.

Rapidly increasing healthcare costs, advances in big data platforms, and wide availability of rich healthcare data have all led to substantial interest in new solutions for predictive modeling in healthcare. Some of these models are in predicting total healthcare costs, risk scoring and management, identifying potential high cost individuals, evaluating expected population costs for contract pricing and premium setting, and managing chronic disease progression efficiently.

Currently, in most solutions that are both task-specific and custom-made for populations with particular characteristics, modifications to either or both of these aspects would be time consuming. Often, modifications of the system would require significant changes to and testing of the methodology. Additionally, the Society of Actuaries have compared a variety of commercial models marketed by various vendors. They are all focused on risk scoring but may differ in particular focus in isolated areas, including (a) business problems they address, (b) the type of data they use, for example, diagnostic (DX), prescription (RX), procedure, prior costs, or some combination thereof, and (c) the temporal ranges for input data and output costs. It would be advantageous if a solution existed that could work with any type of these datasets with varying temporal ranges. Additionally, the ability to modify the solution with relatively more ease would be advantageous, as the significant time and resource investment in modifying the existing systems is undesirable in many cases, especially the fast-paced, ever-changing landscape of the healthcare industry.

### SUMMARY

Embodiments of the present disclosure may be directed toward a method for generating predictive data. The method comprises building a trained configurable predictive pipeline, based on a set of historical training data for a set of training members and identifying a set of member-level features. The features may be identified based on empirical testing of the set of historical training data. A set of predictive data for each member in the set of testing members is generated using the trained configurable predictive pipeline and a set of historical test data. A predictive report can then be generated for the set of predictive data for each member.

This illustrates an improvement of the current technology, in particular in regards to the member-level granularity of the predictions for a set of members, which provides for more accurate, useful predictions. Additionally, the predictive pipeline is configurable, as described herein, which allows for a reduced time and resource investment in using and reusing the pipeline for varying datasets. In this way, the pipeline may be used in an ‘on-demand’ capacity, updating and retraining based on new, incoming data, without the requirement of a full rebuild to accommodate new datasets.

Embodiments of the present disclosure may be directed toward a system for generating predictive data. The system may comprise a training module comprised to build a trained configurable predictive pipeline, based on a set of historical training data for a set of training members. A feature identification module may be configured to identify a set of member-level features based on empirical testing of the set of historical training data. A prediction module may be configured to generate a set of predictive data for each member in the set of testing members using the trained configurable predictive pipeline and a set of historical test data for the set of testing members. The predictive data may comprise predictions for each of the set of member-level features. The system may further comprise a reporting module configured to generate a predictive report for the set of predictive data for each member in the set of testing members.

Various embodiments of the present disclosure may be directed toward the method described herein, where the building comprises a set of steps. The steps may include training the configurable predictive pipeline using a first subset of the historical training data and predicting a predictive second subset of the set of historical data using the pipeline. The method may include comparing the predictive subset of the historical training data with a second subset of historical training data and modifying weighting in the configurable predictive pipeline based on the comparing. This improvement of the current art allows for accurate predictions using unique, modifiable subsets of client data, including current data subsets. This way, a pipeline can generate predictions and generate them using updated (e.g., new monthly) data.

Additional embodiments may be directed toward the curation of data, including data standardization and addressing temporal drifts in population. Data curation prior to building the pipeline may allow for the use of more diverse sets of data than is currently available. Moreover, data curation may provide for effective use of high-dimensional data, including complex patient data, as described herein.

Embodiments of the present disclosure may be directed toward a computer program product comprising a computer readable storage medium having program instructions embodied therewith, wherein the computer readable storage medium is not a transitory signal per se, the program instructions executable by a processor to cause the process or to perform a method. The method comprises building a trained configurable predictive pipeline, based on a set of historical training data for a set of training members and identifying a set of member-level features. The features may be identified based on empirical testing of the set of historical training data. A set of predictive data for each member in the set of testing members is generated using the trained configurable predictive pipeline and a set of historical test data. A predictive report can then be generated for the set of predictive data for each member.

This illustrates an improvement of the current technology, in particular, regarding the member-level granularity of the predictions for a set of members, which provides for more accurate, useful predictions. Additionally, the predictive pipeline is configurable, as described herein, which allows for a reduced time and resource investment in using and reusing the pipeline for varying datasets. In this way, the pipeline may be used in an ‘on-demand’ capacity, updating and retraining based on new, incoming data, without the requirement of a full rebuild to accommodate new datasets.

Embodiments of the present disclosure may be directed toward a method for generating predictive healthcare cost data. The method may comprise building a trained configurable predictive healthcare cost pipeline for a set of training members. The set of training members are a set of members enrolled in a particular health insurance program. A set of member-level features may be identified based on empirical testing of the set of historical patient training data for the set of training members. A set of predictive data for each member in a set of testing members may be generated using the trained configurable predictive healthcare cost pipeline and a set of historical patient test data for a set of testing members in the testing set. A set of predictive data for each member may be used to generate a predictive report. In embodiments, the predictive report may comprise a set of medical risk factors, a predictive risk score, and a set of predictive costs.

Embodiments of the present disclosure may be directed toward a system for generating predictive healthcare cost data that comprise a computer readable storage medium with program instructions embodied thereon and one or more processors configured to execute the program instructions to perform a method. The method may comprise building a trained configurable predictive healthcare cost pipeline for a set of training members. The set of training members are a set of members enrolled in a particular health insurance program. A set of member-level features may be identified based on empirical testing of the set of historical patient training data for the set of training members. A set of predictive data for each member in a set of testing members may be generated using the trained configurable predictive healthcare cost pipeline and a set of historical patient test data for a set of testing members in the testing set. A set of predictive data for each member may be used to generate a predictive report. In embodiments, the predictive report may comprise a set of medical risk factors, a predictive risk score, and a set of predictive costs.

The above summary is not intended to describe each illustrated embodiment or every implementation of the present disclosure.

### BRIEF DESCRIPTION OF THE DRAWINGS

The drawings included in the present application are incorporated into, and form part of, the specification. They illustrate embodiments of the present disclosure and, along with the description, serve to explain the principles of the disclosure. The drawings are only illustrative of certain embodiments and do not limit the disclosure.

FIG. 1 depicts a diagram of a system for generating predictive reports, according to embodiments.

FIG. 2 depicts a flow diagram of a method for generating predictive reports, according to embodiments

FIG. 3 depicts a diagrammatic flow for a training and testing of a predictive pipeline 300, according to embodiments.

FIG. 4 depicts a flow diagram of a method for building a predictive pipeline, according to embodiments.

FIG. 5 depicts a visual representation of the features of variable member history durations used in training and testing the model.

FIG. 6 depicts a block diagram of a method for predicting healthcare data, wherein the method is organized into a set of APIs, according to embodiments.

FIG. 7 depicts of an example UI of a system management module for selecting and verifying previously executed APIs, according to embodiments.

FIG. 8 depicts an example of compressed data representation, according to embodiments.

FIG. 9 depicts an example graph and chart, for use in handling outliers, according to embodiments.

FIG. 10 depicts a block diagram of a particular embodiment of a configurable predictive pipeline architecture, including the modules depicted herein, according to embodiments.

FIG. 11 depicts a sample UI of an individual report, according to embodiments.

FIG. 12 depicts a high-level block diagram illustrating an example computer system that can be used in implementing one or more of the methods, tools, and modules, and any related functions, described herein, according to embodiments.

FIG. 13 depicts a cloud computing environment according to an embodiment of the present invention.

FIG. 14 depicts abstraction model layers according to an embodiment of the present invention.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

Aspects of the present disclosure relate to computer systems, and more particular aspects relate to predictive pipeline applications in computer systems. While the present disclosure is not necessarily limited to such applications, various aspects of the disclosure may be appreciated through a discussion of various examples using this context.

Rapidly increasing healthcare costs, advances in big data platforms, and wide availability of rich healthcare data have all led to substantial interest in new solutions for predictive modeling in healthcare. One of the goals of AI-based healthcare solutions is to provide accurate predictions of healthcare costs for new members. Some applications of predictive modeling are in predicting total healthcare costs, risk scoring and management, identifying potential high cost individuals, evaluating expected population costs for contract pricing and premium setting, and managing chronic disease progression efficiently.

It may be advantageous to provide accurate predictions as early as possible. It may also be helpful to identify high-cost individuals with reliable accuracy. At times, this may be a complicated problem, with implications that may impact several stakeholders including, for example, insurance companies (i.e., “payers”), hospitals (i.e., “providers”), employers, government entities, and others.

Research in predictive modeling for healthcare may be along one of two main fronts. Academic research has focused mostly on the development and evaluation of predictive learning methods. This may be just one component of a complete real-world solution. Some examples of this academic approach include regression-based methods such as, but not limited to, linear regression, least absolute shrinkage and selection operator (LASSO), or logistic regression. Other examples may include tree-based methods such as decision trees, random forests, boosted trees, and clustering approaches. Platforms have been proposed that may compare many predictive learners in parallel using electronic health records.

On the other hand, commercial products have been developed and may focus on real-world applications. Many commercial models are proprietary, and they may be developed for specific predictive statistics and well-defined patient populations. For example, multiple studies have been commissioned by the Society of Actuaries in which a variety of commercial models have been compared. Commercial models may be focused on risk scoring, but they may differ from one another in terms of (a) the business problems they address, (b) the type of data used (e.g., diagnostic, prescription, procedures, prior costs, or some combination thereof), and (c) the temporal ranges for input data and output costs.

Since many of the commercial approaches are task-specific and custom-made for populations with particular characteristics, changing one or both of these could be time consuming and may require significant modification and testing of the methodology. However, a ‘one size fits all’ approach that does not account for population characteristics may not adapt well to new populations and may not be robust.

In embodiments, a modular, configurable, and extensible big data system for healthcare cost prediction like the one proposed herein may help address these issues. A platform that may be easily configured and adapted to rapidly develop predictive healthcare solutions for different populations and predictive tasks could be used by health insurance companies, risk-sharing providers, and self-insured employers. The system, method, and computer program product for generating healthcare cost predictive data may be first built through training, tested, and then used to generate the predictive data. The configurable predictive pipeline may be built based on a set of historical training data for a set of members. As used herein “members” may include individuals or groups of individuals relevant to predictions, for example, an individual who is a part of a particular health insurance plan. Prior to use, the data may be standardized, as described herein, and selected based on user preferences, or as is appropriate for the particular client, and desired prediction output. In embodiments, the historical training data may be a set of patient data collected over a predetermined period of time in the past (e.g., pre-existing data). For example, a system may use data for a particular set of members enrolled in a plan from January 2015-January 2016. The patient data used here could include high-dimensional data, as described herein.

A high level of configurability may support flexible feature creation, predictive learning, and report generation after prediction. By providing fine-grained control to the user over a time period for inputs and outputs, training and scoring, the system may be trained on-demand. As described herein, ‘on-demand’ training can mean the system uses the most recent and most appropriate data to produce more accurate models that can be deployed immediately. Distributional changes between training and testing can be corrected by a population shift capability. Besides providing accurate predictions, the system may also employ sensitivity analysis to identify key risk drivers behind the predictions, making results more interpretable and actionable.

An end-to-end pipeline, according to embodiments described herein, may be automated and include a web-based management module that may visualize results and allow for reuse of partial outputs and models from previous runs (e.g., data sets). Several modules of the pipeline may support parallelization. In embodiments, feature selection (e.g., selection of member-level features) may be employed before and during predictive learning training to exploit the rich information in high-dimensional data, while keeping computational complexity in check. As used herein, high-dimensional data may be characterized as such based on the multi-faceted nature of the data. For example, when using patient data described as ‘high-dimensional’, data for each patient may include tens of thousands of features, or more. For example, the patient data could include a set or sets of demographic data to identify the patient (e.g., age, biological statistics, geographical data, or other relevant data). The patient data could also include a set or sets of data describing a particular diagnosis course or courses relevant to that patient. The data could also include a set of data describing drug or medication courses as well as procedural courses. For example, the medication data could describe historically, currently, and predictively a set of medications prescribed to the patient for a particular diagnosis, or it could describe the medication set in its entirety for a patient.

In embodiments, the system may build the configurable predictive pipeline by first training the pipeline using a first subset of the historical training data. For example, the first subset of historical training data may be patient data for the set of members from January 2015-September 2015. Using the predictive pipeline, the system may then predict a second subset of the historical training data. For example, the system may predict various outputs for the set of members from October 2015-January 2016. The system may then compare the predicted outputs from the historical training data with a second subset of the historical training data, and adjust the configurable predictive pipeline accordingly. For example, the predictive patient data from October 2015-January 2016 may vary from the actual, historical patient data for the particular members from October 2015-January 2016. Differences may result in modifications to weighting within the predictive pipeline. This building and training may be an iterative process, repeated for each pipeline as many times as possible, until a predetermined level of accuracy has been achieved.

The system may then identify a set of member-level features, based on empirical testing of the set of historical data for the set of members. In embodiments, member-level features may be pre-selected by a user or client using empirical testing and estimates of significance, as described herein, or they may be forcibly included or excluded, regardless of their significance. For example, member level features may include features such as diagnosis, procedures, and drug codes that are correlated (to a determined level of significance) with the total claims cost. In embodiments, the system may then test the predictive pipeline, using a set of historical test data for the set of members. The historical test data, like the set of historical training data used in the initial build, may be high-dimensional data and include patient data relevant to various aspects of patient health and care. In embodiments, the testing may occur by running the set of historical test data through the predictive pipeline, to generate a set of predicted outputs for the set of members. The predicted outputs may comprise predictions for each of the set of member-level features. The system may then generate a predicted outputs for each member based on the set of predictive data. In embodiments, the report may be transmitted to another system for use (e.g., a health insurance system, a hospital system, or another appropriate system), to a user interface for communication to the user (e.g., to a client in real-time), or to another entity for an approved use.

The processes described above may be executed over any number of modules, in a variety of organizational structures; for ease of understanding, specific modules may be described herein, as follows.

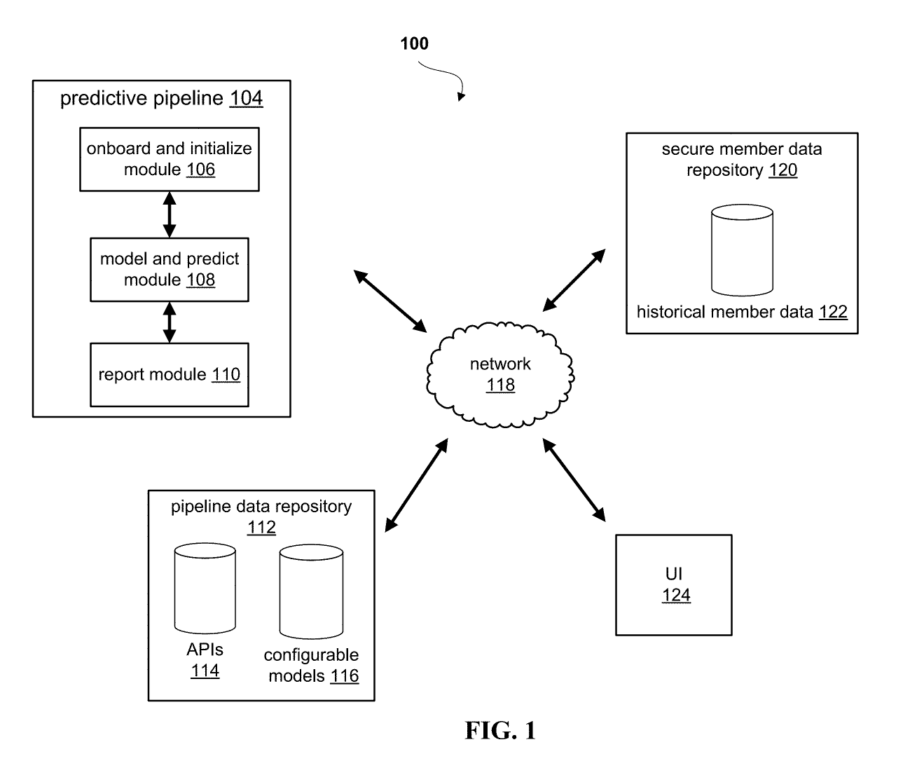

FIG. 1 depicts a diagram of a system 100 for generating predictive reports, according to embodiments. The predictive pipeline system 104 depicted here can comprise at least one computer processor circuit, which can be configured to predict healthcare costs for users, consistent with embodiments. In embodiments, high-dimensional data, including historical member data 122, may be accessed from a secure member data repository 120 over a network 118. In embodiments, this data repository may be a commercial claims database such as MARKETSCAN, as described herein, or another appropriate database or data source. In embodiments, the predictive pipeline system 104 may be provided with the data, rather than access the data. The predictive pipeline system 104 may comprise a set of modules including an onboard and initialize module 106, a model and predict module 108, and a report module 110. The predictive pipeline system 104 may comprise more or fewer modules than the modules depicted herein. The onboard and initialize module 106 of the predictive pipeline system 104 may receive the data from secure member data repository 120. In embodiments, the data received from the secure member data repository 120 (e.g., historical member data 122), may be raw, high-dimensional data. The onboard and initialize module 106 may sort, select, and further curate the data as described herein. The curated data may then be passed to a model and predict module 108, which may build the model using the curated data through an iterative training process, and, using test data, predict a set of results relative to a set of members identified in the historical member data 122.

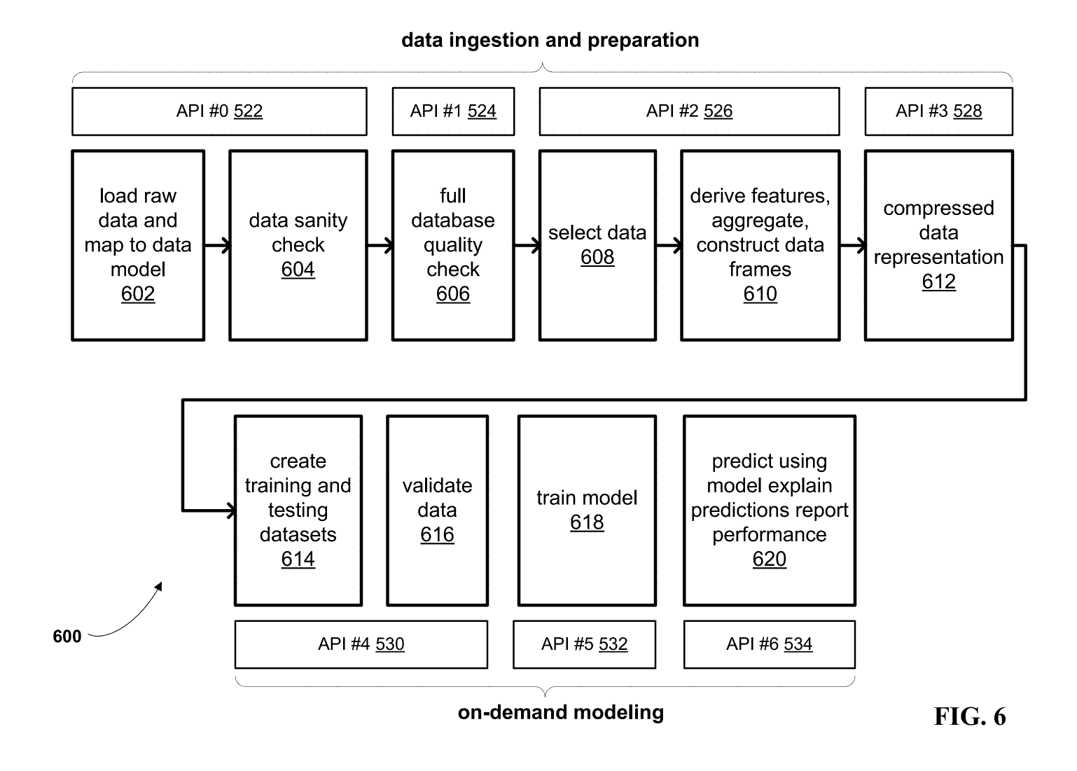

In embodiments, the configuration data and other performance data generated by the model and predict module 108 may be stored in a pipeline data repository 112. In embodiments, this data may be transmitted over a network 118, for storage in the pipeline data repository 112. In some embodiments, the pipeline data repository 112 may be a part of the predictive pipeline system 104 or it may be communicatively coupled as depicted, existing in a remote location. The pipeline data repository may be organized in several fashions, one of which is depicted at FIG. 1, including the division of an application programming interfaces (APIs) database 114 and a configurable models database 116. In embodiments, the APIs database 114 could comprise API configurations for each of the APIs associated with the predictive pipeline system 104 (for more on APIs and the predictive pipeline system 104, see, e.g., FIG. 6). The configurable models database 116 could store configuration data or other data from the training and testing of the pipeline (e.g., by the model and predict module 108). This data could be stored following each iteration of the training, after a particular level of accuracy has been reached, following a prediction report generation, or at another time.

The predictions, as well as other data described herein (e.g., performance results), may be passed to the report module 110. The report module 110 may transmit a generated report or set of reports over the network to a user interface (UI) 124 for display to a client. In embodiments, the report may be sent to a printer to produce a physical copy, a database for storage and future access, to another system for use, or transmitted to another entity, as deemed appropriate.

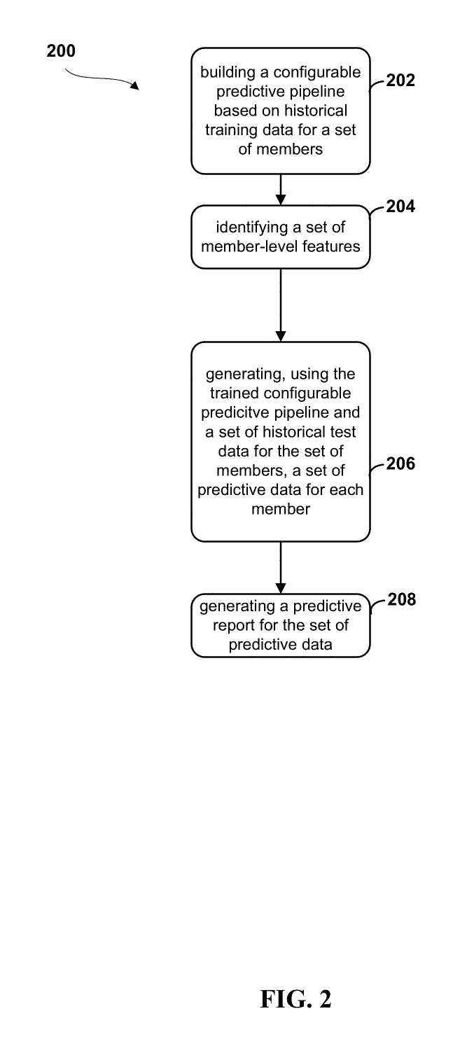

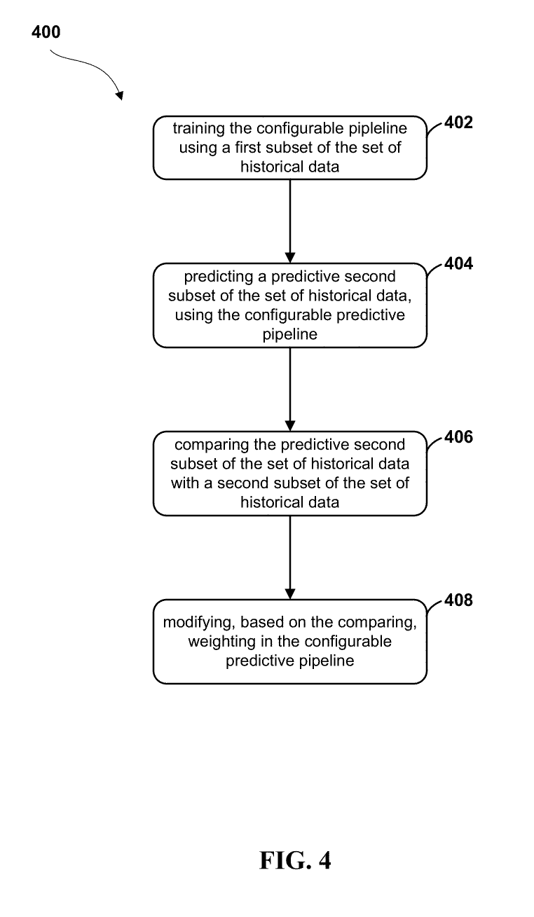

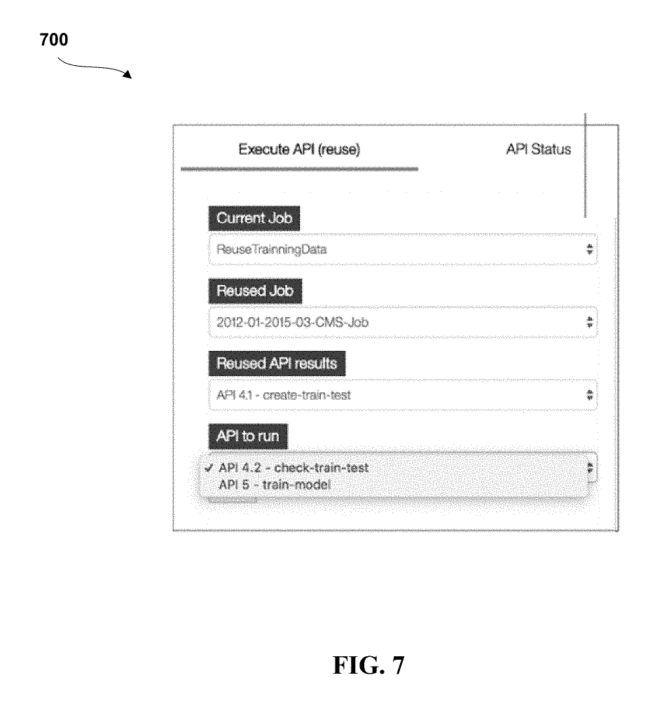

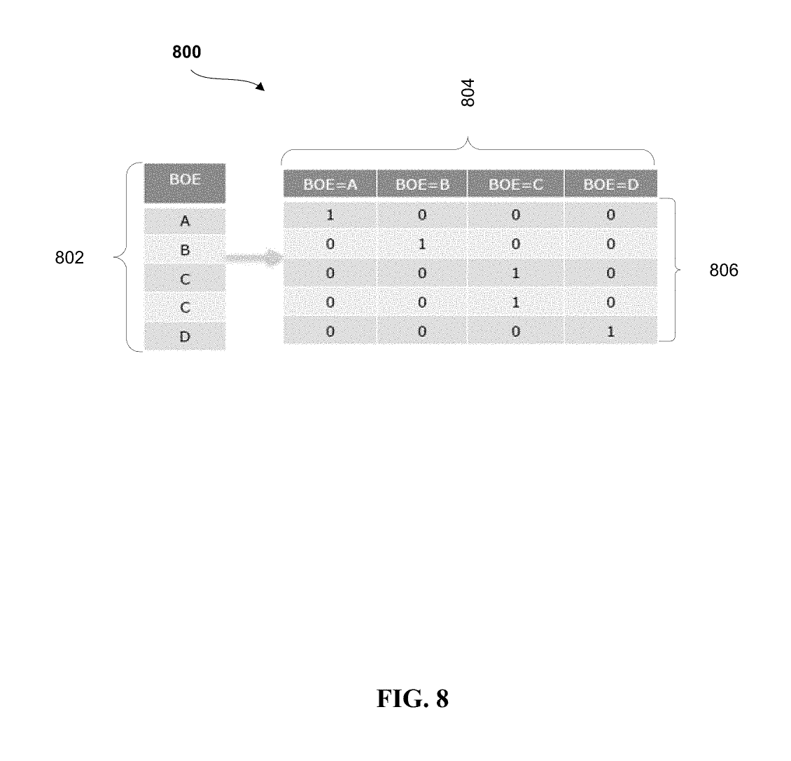

FIG. 2 depicts a flow diagram of a method 200 for generating predictive reports, according to embodiments. The method 200 may begin when a configurable predictive pipeline (e.g., predictive pipeline 104 of FIG. 1), is built based on historical training data for a set of members, at 202. The predictive pipeline may be built according to known methods, or as described at, for example, FIG. 4. In embodiments, the building and training of the model may include data curation, standardization, and characterized representation, as described herein, and specifically, for example, at FIGS. 7-8.

Based on the data generated by the configurable pipeline, a set of member-level features may be identified, at 204. Features may be determined for various levels of granularity, according to settings, the data, and/or client preferences, with member-level preferences being a highly granular level of feature selection. In embodiments, features may be identified based on their significance to a data set (e.g., to a set of member data). This significance-level identification may be performed by studying a marginal correlation of the variables to the target and estimating their significance using empirical testing. One example of empirical testing that may be used is permutation testing. In embodiments, features may also be selected irrespective of their significance levels. For example, features determined to be highly significant may be forcibly included, and vice versa. In embodiments, features may be selected based on a hybrid of significance-relevant determinations and significant-irrelevant determinations.

In embodiments, the trained predictive pipeline may be fed a set of historical test data for the same set of members used to train the model. In embodiments, the trained predictive pipeline may use historical test data for a different set of members than those used to train the model. The system may generate a set of predictive data for each member, using the pipeline, at 206. In embodiments, the system may generate a predictive report for the set of predictive data, per 208. In embodiments, local post hoc explanations for predictions may be provided. In embodiments, simple surrogates, for example, linear models, may be used for local explanations. Reports that quantify the performance of the trained model (using, e.g., historical data with known targets), along with predictions and the explanations may be created. For example, the report may include risk factors, predictive risk scores, predictive costs, and other relevant data.

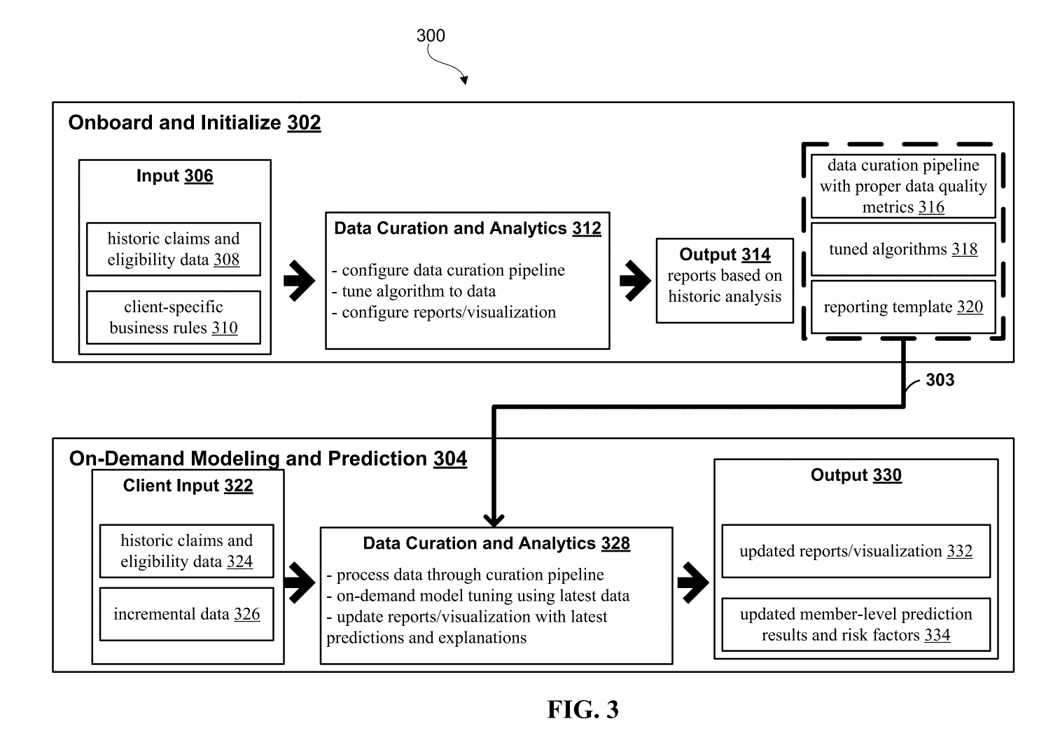

FIG. 3 depicts a diagrammatic flow for a training and testing of a predictive pipeline 300, according to embodiments. In embodiments, the predictive pipeline 300 may be predictive pipeline 104 of FIG. 1, as well as the pipeline referred to in the method 200 of FIG. 2. Predictive pipeline 300 may be conceptualized in two distinct modules, an onboard and initialize module 302 and an on-demand modeling and prediction module 304. Together, they may form a configurable predictive pipeline 300. The first phase, using the onboard and initialize module 302, may comprise the utilization of an input engine 306, a data curation and analytics engine 312, and an output engine 314. In embodiments, the input engine 306 may receive historical claims and eligibility data 308 and client-specific business rules 310. In embodiments, this data may be securely pulled from databases, or accessed or received in another suitable way. This data may be input to the data curation and analytics engine 312, which may use this input to build and train the predictive pipeline 300. Specifically, the data curation and analytics engine 312 may configure a data curation pipeline to, for example, further select relevant data from the inputs, and derive additional features that may be useful in modeling. The engine may also aggregate features at a member level for a specific temporal resolution. In embodiments, the algorithms may be tuned to the specific data set by data curation and analytics engine 312. In embodiments, the engine may also handle configuration of reports and visualization, including formatting, configuration of UI presentation (e.g., data fields to be displayed, level of detail to be presented in the report, format of various data in the report, for example, graphical representations), or other visualization-based concerns. In embodiments, the data curation and analytics engine 312 may generate a reporting template, for use by the on-demand modeling and predictions module 304. An output engine 314, as part of the onboard and initialize module 302, may run historical data, as described herein, through the pipeline and generate reports based on the historical analysis.

In embodiments, the on-demand modeling and predictions module 304 may be informed by modeling and data from the onboard and initialize module 302, as depicted by arrow 303. In embodiments, proper data quality metrics for the data curation pipeline 316, a set of tuned algorithms 318, and a reporting template 320 (e.g., the reporting template generated by the data curation and analytics engine 312) may be generated. In embodiments, this data (e.g., data curation pipeline with proper data metrics 316, the set of tuned algorithms 318, and the reporting template 318) may be provided to and for use by a data curation and analytics engine 328 of the on-demand modeling and prediction module 304.

In embodiments, the on-demand modeling and prediction module 304 may be used in a second, client-specific phase of prediction report generation. In embodiments, a client input engine 322 of the on-demand modeling and prediction 304 may input historical claims and eligibility data 324 and incremental data 326. In embodiments, the historic claims and eligibility data 324 and the incremental data 326 may be high-dimensional data and may be historical test data, as used herein. This data may be input to and processed by the data curation and analytics engine 328, which may also receive and incorporate the pipeline data from the onboard and initialize module 302. The data curation and analytics engine 328 may process data through curation of the pipeline, and may tune the model ‘on-demand’ using the latest data (e.g., historic claims and eligibility data 324 and incremental data 326 received from the client input engine 322). In embodiments, the data curation and analytics engine 328 of the on-demand modeling and prediction 304 may tune the model in a variety of ways. For example, the engine may incorporate noise robustness using, for example, robust loss functions including Huber loss functions or quantile losses. Noise robustness may also be incorporated using partitioning models based on the target cost quantiles, as described further herein. The base learning approach used in the modeling may include a combination of standard linear and non-linear machine learning methods. Compositional learning approaches (e.g., deep learning) may also be used, along with feature selection. These various learning approaches may be combined and modified to improve predictions. In embodiments, the data curation and analytics engine 328 may also compensate for distributional drift and cost inflation. In embodiments, distributional drift between the training and testing data may be compensated for by a re-weighting of the training data distribution to match the test data distribution. In embodiments, temporal drifts in population may be accounted for in a principled manner. For example, a training set of patient data from New York may be used to train a model for testing and prediction of data for a set of patients in California. The data may be rerated in order to accommodate for variances between features in patient data between the two geographies, in order to provide for accurate training of a model, despite the differences in populations. In this way, the model can be built and trained using a diversity of data, and to provide for accurate predictions despite variances in datasets.

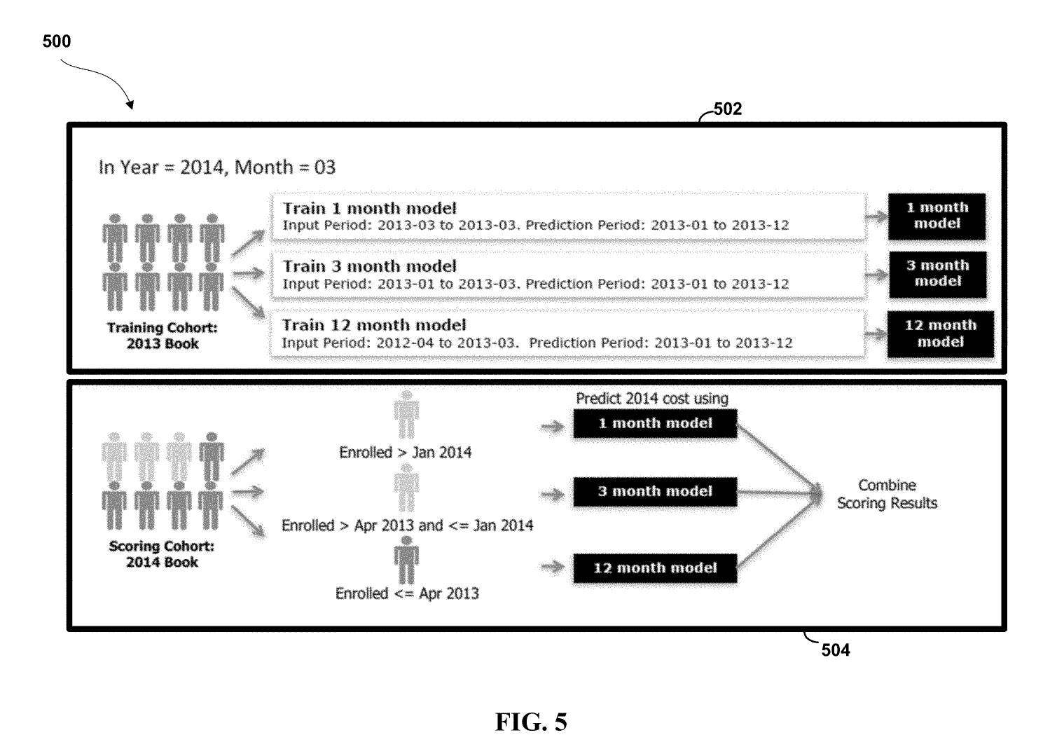

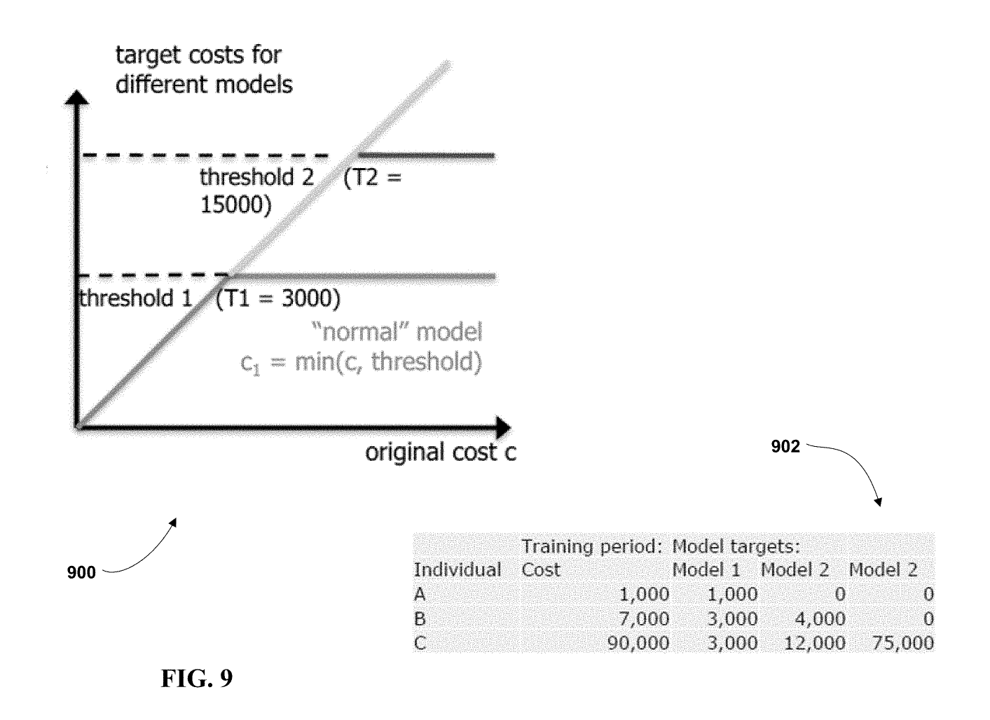

Cost inflation may be compensated for by learning and applying an inflation model from historical data. Additionally, varying member history may be factored into the on-demand training, including the use and training of multiple predictive models for members with varying history, to ensure that large amounts of data are being used in training. Further example of the use and combination of hybrid training and prediction models for differing member history is shown at FIG. 5. An example approach of compensation for outlying members that may be used in training and prediction with the pipeline is shown at FIG. 9.

In embodiments, like the data curation and analytics engine 312 of the onboard and initialize module 302, the data curation and analytics engine 328 may also process data through the curation pipeline and handle reports and visualization. As this model is working with on-demand training, using client data for testing (i.e., predicting) results, the system may generate reports and visualizations with a latest set of predictions and explanations. The on-demand modeling and prediction module 304 may process data and generate a particular set of outputs, handled by an output engine 330. In embodiments, this output can include updated reports and visualization 332 and updated member-level prediction results and risk factors 334. In embodiments, this output may be a predictive report for a particular member or set of members in a health insurance group. In embodiments, the predictive report could be configured to present data on membership on various levels of granularity. For example, predictive data could be presented on a member-level basis. It could also be presented in reports at a cohort level, as defined by an administrator, client, or in another manner. The report could also comprise a combination of granularity levels, in order to provide for a more comprehensive portrait of health care cost prediction. The output engine 330 may also be responsible for communicating one or more of these outputs to a specified user interface within the system, to another system, to a database for storage, or to another entity, as is deemed appropriate for the client and data use.

FIG. 4 depicts a flow diagram of a method 400 for building a predictive pipeline, according to embodiments. The method 400 may be an example of step 202 at FIG. 2, wherein a predictive pipeline is built and trained according to a set of historical data. The process may be carried out of a set of one or more processors, in a cloud computing environment, or in another way. The method 400 may begin when a configurable pipeline (for example, predictive pipeline 300 of FIG. 3) is trained using a first subset of a set of historical data. As described herein, the historical data could be a set of high-dimensional patient data, where the patients are members of a particular healthcare population. For example, the set of historical data could be all available patient data for patients who joined a particular health insurance group in December 2015 (hereinafter “December 2015 members”). A first subset of the data could be patient data for the December 2015 members between January 2016 and July 2016. In embodiments, this data could be used to train the model (e.g., adjusting the weighting within the predictive modeling).

In embodiments, a predictive second subset of historical data could be generated using the trained configurable predictive pipeline, per 404. For example, the trained pipeline could be used to generate a set of data for the December 2015 members between August 2016 and December 2016. The predictive second subset of the set of historical data could then be compared with the actual second subset of the set of historical data, per 406. For example, the actual data for the December 2015 members could then be compared with the generated predictive data. Based on the comparison, weighting within the configurable predictive pipeline may be modified, per 408, to improve the accuracy of the pipeline. In embodiments, this may be an iterative process, repeated as many times as required by a preferred accuracy measure (e.g., an accuracy threshold or other standard).

FIG. 5 depicts a visual representation 500 of the features of variable member history durations used in training and testing the model, according to embodiments. The figure is divided into a training phase 502 and a testing phase 504.

In embodiments, a training phase 502 may include a set of sample data for members (“training cohort 2013 book”) at a particular year, “2014”, and month “03”. The cohort may be divided into three groups, as depicted in the three rows, to include a “train 1-, train 3-, and train 12-month model”. Three different models may be created, as described herein, to capture and utilize a dataset for members with histories varying from one month to a year (or more). A first model (e.g., the first row) may use input data from a single month, March 2013, (e.g., “input period=2013-03 to 2013-03). The model may then be used to predict from January 2013 to December 2013 (e.g., “prediction period: 2013-01 to 2013-12”). A second model (e.g., the second row) may use input data from three months, from January 2013 to March 2013, for training (e.g., “input period: 2013-01 to 2013-03”). The model may then be used to predict from January 2013 to December 2013 (e.g., “prediction period: 2013-01 to 2013-12”). A third model (e.g., the third row) may use input data from twelve months, from April 2012 to March 2013 (e.g., “input period: 2012-04 to 2013-03”). The model may then be used to predict from January 2013 to Decembers 2013 (e.g., “prediction period: 2013-01 to 2013-12”).

A second phase, the testing phase 504, shows how the variable models may be used to score members with different member history. For example, a scoring cohort of members, for whom cost predictions will be made (e.g., “scoring cohort: 2014 book”), may be divided into three groups, based on the data available for them as members of a particular healthcare program, or other membership. As depicted, a first group may consist of a single member or member set (e.g., the top figure, “enrolled>January 2014”). This member set may have been enrolled for a month or less, and this member set's costs may be predicted using the 1-month model. In embodiments, a second member set of the scoring cohort may be been enrolled between April 2013 and January 2014, and the data associated with this membership may be processed using the generated 3-month model. Finally, a third member set that was enrolled before or since April 2013 (e.g., the bottom figure, “enrolled<=April 2013”), may be processed using the 12-month model. The results from these three subsets of the scoring cohort may then be combined, as described herein, in order to provide an accurate cost prediction. In this way, members with less member history, for example, the first two figures processed by the 1-month and 3-month models, respectively, can still be accounted for and their relevant data captured in the process.

FIG. 6 depicts a block diagram of a method 600 for predicting healthcare data, wherein the method is organized into a set of APIs, according to embodiments. In embodiments, the method 600 may comprise steps 602-620. Each of steps 602-620 may be separated into particular APIs, for example API #0-6, numbered 522-534, respectively. Conceptualizing each step or groups of steps into a particular application programming interface (API) or APIs is one way in which the predictive pipeline, e.g., predictive pipeline 104 of FIG. 1, may provide for configurability and ‘on-demand’ training. In embodiments, the steps of method 600 depicted at 602-620 may be carried out over one or more processors, and may, in combination with the associated hardware (e.g., one or more processors) be referred to as the configurable predictive pipeline. Each API may have a particular input, a particular output, and a particular configuration or set of configurations. These may be dynamic, changing with each client, each dataset, each iteration of training, or as appropriate. This can provide for modular configurability, in that new or different portions of the pipeline that need to be modified or reconfigured (e.g., based on new or differing data sets (e.g., changes in population) or new or different targets, desired results, tasks, etc.) could be isolated and reconfigured independent of the pipeline as a whole.

In embodiments, the first set of APIs (API #0-3 522-528) can be used to provide the data ingestion and preparation phase of the pipeline, and the second set of APIs (API #4-6 530-534) may comprise the on-demand modeling phase of the pipeline. These two phases, data ingestion and preparation phase and on-demand modeling phase, may be analogous to the onboard and initialize module 302 and on-demand modeling and prediction module 304, each of FIG. 3, respectively. The method 600 may begin when raw data is loaded to the system and mapped to a model, per 602. A data sanity check, 604, may then be performed. In embodiments, this check can occur as an estimation of statistics for the data, to ensure the standardization and pipeline is properly aligned with the data. As depicted, configurations for these two steps, 602 and 604, may be saved for use at API #0 522. Next, the system may perform a full database quality check, per 606; this process may be performed at API #1 524. The next steps of data selection, per 608, and feature derivation and aggregation, as well as construction of data into frames, per 610 may be performed at API #2 526. As described herein, this may include selection of key features (e.g., at an individual or member level, or at another level of specificity), the organization of data, and other forms of data curation.

The data may be a mixture of continuous and categorical variables. The categorical variables with a large number of categories may then be represented using a compressed representation that can be directly manipulated by software, for example at step 612. In embodiments, the various configurations and processes associated with improved simplicity in data representation may be saved and relative to API #3 at 528. One example, depicted at FIG. 8, may include the use of dummy coding and sparse matrix representation.

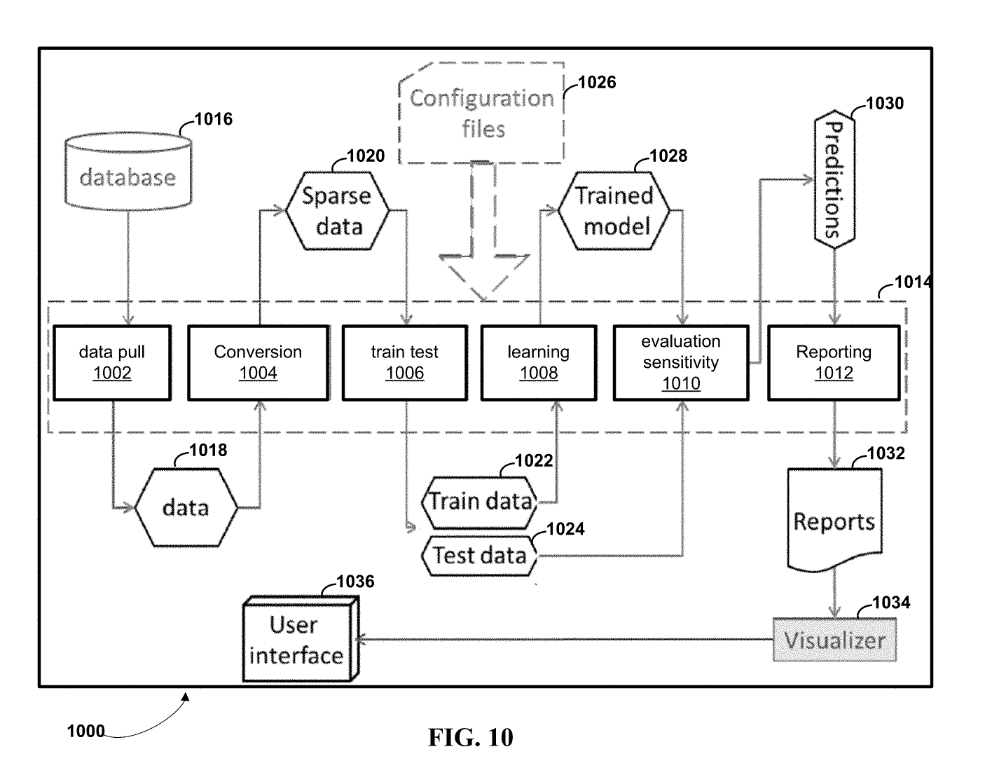

The remaining steps, from steps 614-620, may be considered part of the on-demand modeling phase of the method 600 for generating predictive healthcare data. At step 614, datasets of training and testing data may be created. For example, the datasets may be created using the curated data from the previous steps (602-612). This data in its raw form may have been high-dimensional data, which may be historical patient data, e.g., from a database or other source, as described herein and in the particular embodiment following FIG. 10. At 616, the data can be validated using methods known in the art. These processes may be umbrellaed under a same API, API #4 530. The validated data can then be used to train the model, per 618, as described herein. In embodiments described herein, the training of the model, step 618, may be analogous to the building step, wherein historical training data is used to tune the model. The input, output, and configuration data for this step may be generated and stored relative to API #5 532. Finally, the built and trained model may be tested, and the testing dataset (e.g., from step 614) may be used to predict costs for the dataset, per 620. The model may be used to generate a report, as well as generate an explanation of the performance and performance (e.g., accuracy) statistics of the data run. This final step may be modularized under API #6 534.

FIG. 7 depicts of an example UI 700 of a system management module for selecting and verifying previously generated configurations relative to particular APIs, according to embodiments. The UI 700 may be used to recall input, output, and configurations, for example, relative to specific APIs as depicted at FIG. 6. As described herein, the storage of configurations of portions of the pipeline may provide for a particular level of granularity in the configurability and reusability of various trained aspects of the pipeline. In this way, the pipeline may embody elements of a ‘plug-and-play’ pipeline, where client data may be provided, and relevant portions modified, without the need for an entirely new pipeline to be generated for each client and for each data set.

FIG. 8 depicts an example of compressed data representation 800, according to embodiments. For example, the compressed data representation 800 may include a first chart 802 labeled “BOE”. Prior to its use as training or testing data, raw client data (e.g., likely high-dimensional data) may be curated in any number of several ways. One way can be through the use of efficient data representation. The data can be a mixture of continuous and categorical variables, and as stated herein, the categorical variables with a large number of categories can be efficiently represented using a compressed representation (e.g., compressed data representation 800), which can then be directly used and manipulated by software. In the example provided, BOE may be a categorical feature, of which there may be many. Each row in 802 under BOE may indicate a different aspect or variable within the BOE category. The various BOE options may then be dummy coded into a sparse matrix, with each column 804 indicating a different variable within BOE, and each row 806 containing dummy coding (e.g., the presence or absence) of the particular variable in the given dataset. The sparse matrixes created in this manner can then be more effectively used by the model for training and testing.

FIG. 9 depicts an example graph 900 and chart 902, for use in handling outliers, according to embodiments. Graph 900 depicts a model for handling outlier data, for example, high cost patients, in an organized fashion. Table 902 depicts a table for a model for handling high-cost patients in a principled manner. In embodiments, when training, the total cost (e.g., target) for each individual (e.g., patient) is broken up with each piece assigned to a corresponding model. When scoring (e.g., testing or making predictions), the outputs of all models are summed to arrive at the prediction. Thresholds are not used in the scoring.

For example, graph 900 depicts a graph, with the x-axis representing an original cost “c” and a y-axis representing target costs for different models. The target cost for the first model may be equal to the lesser of the original cost or a first threshold T1. A residual may be computed as the difference between the original cost and the first target cost. The target cost for the second model may then be determined as the lesser of the residual or a second threshold T2. A second residual may be computed as the difference between the first residual and second target cost. This second residual may then become the target cost for a third model.

FIG. 10 depicts a block diagram of a particular embodiment of a configurable predictive pipeline architecture 1000, including the modules depicted herein, according to embodiments. According to embodiments, the architecture 1000 may comprise the configurable predictive pipeline (CPP) 1014, as well as a set of inputs and outputs 1016-1032 of the components. In embodiments, the rectangular blocks 1002-1012 may depict components of the CPP 1014, and the hexagonal blocks 1018-1024 and 1028-1030, as well as configuration files 1026 may depict the inputs and outputs of the components. In embodiments, a visualizer 1034 and the user interface 1036 may be considered a part of the CPP 1014, or may be separate entities, as depicted herein. A particular embodiment of the CPP 1014 and CPP architecture 1000 is described below.

This embodiment may include an end-to-end solution stretching from a claim or set of claims by a user and enrollment data to reporting and visualizations of results, including data curation, learning, and prediction modules in-between. In embodiments, the system may be highly-configurable. For example, configuration parameters may pertain to both the given data set as well as to the function of the module, for example feature sets used in modeling. Thus when the system is run on a new data source or the experimental design changes, the configuration files may be the only files that need to be modified by the user, allowing the actual modules to remain unaltered. Overall, the configurability and modularity may provide substantial flexibility not only in re-using existing functionalities but also in creating new functionalities with a “plug-and-play” approach.

In particular embodiments, the set of modules may include those depicted in FIG. 10, with the first two modules, a data pull module 1002 and a conversion module 1004, curating data obtained from the user's database. A first module, the data pulling module, 1002, may be a data retrieval module that pulls claims and enrollment data from the database tables onto a local machine. In embodiments, the data is collected from a time period which may, for example, span several years, and may be aggregated to a common temporal resolution, for example, one month. The temporal resolution may be used in subsequent modules. Additional derived features may supplement existing claims features, for example, condition classifications based on diagnosis codes may be used. The configuration parameters may include database information and metadata, desired data range for the data pull, and specifics on the features to be created.

In embodiments, a conversion module 1004, for example a sparse matrix conversion module, may recode categorical data (e.g., categorical data 1018) into binary ‘dummy variables’ as required for numerical modeling. The module may then convert the binary ‘dummy variables’ to sparse matrices (e.g., sparse data 1020) for effective handling. For example, an insurance product type category like “PPO”, “HMO”, etc. may be coded into features “product_type=PPO”, “product_type=HMO”, etc. which assume values of 1 or 0. For features with thousands of categories such as medical codes, the subsequent sparse matrix conversion dramatically reduces storage and computations in downstream modules compared to a naïve dense representation. The configuration parameters specify the categorical features, and the output may be a set of sparse matrices (e.g., sparse data 1020).

In embodiments, sparse data 1020 may also be a subset of testing or training data, to provide for the on-demand aspect of the modeling. For example, the model may be trained using a set of data for a particular set of members from February 2016 to February 2017. Upon receipt of a new data set, for example, training data for the set of members from March 2017 to April 2017, the system may be retrained using the new dataset. Thus, the model may be used and modified quickly without the need for an entirely new model build. The newly configured model may then be used to generate a new set of predictive data, where the predictions account for the new data. In embodiments, parameters may be adjusted to provide for a pipeline trained according to specific datasets. For example, upon the receipt of the new data (e.g., March 2017-April 2017), the model may adjust to remove the previous month from the pipeline training (e.g., February 2016-March 2016 data may no longer be used to train the pipeline).

In particular embodiments, the next three modules, train test module 1006, learning module 1008, and evaluation sensitivity module 1010, in the system CPP pipeline 1014 may relate to the building and application of predictive models. A train test module 1006 may further aggregate the sparse matrices (e.g., sparse data 1020) from the previous step to create training and testing datasets for the predictive learner. Data may be aggregated over an input period to produce a single feature vector per individual, while the individual's cost (response variable) may be aggregated over the output or prediction period. This module may also select which individuals are included in the training and testing sets (e.g., train data 1022 and test data 1024, respectively), based on cohort inclusion criteria (i.e., coverage conditions), namely the required length of enrollment within the input and output periods. The features to be created, input, output, and runout periods, and coverage conditions may be some key configuration points. Some or all of this data may be received from configuration files 1026, provided to the pipeline by, for example, a system manager, from memory, from another system, from an external source, or in another way.

A predictive learning module, e.g., learning module 1008, may instantiate a multistage predictive learner as a class for ease of use by subsequent modules. A fit method of the multistage predictive learner class may perform predictive learner fitting and optionally feature pre-selection and co-variate shift correction using the set of train data 1022. Additional preprocessing steps such as winsorizing may also be done. The parameters for feature pre-selection, covariate shift correction, and the number and type of stage used in the predictive learning module may be configurable. The module may save the trained learners as files (e.g., trained model 1028).

An evaluation and sensitivity analysis module (e.g., evaluations sensitivity module 1010) may be used to score testing data (e.g., test data 1024) using the trained learner to generate predictions 1030. The module may also perform sensitivity analysis to compute the individual-level cost impacts of selected input predictors. Locations of the trained learner and data and sensitivity analysis parameters may be specified in the associated configuration file 1026. The module may output individual prediction and cost impacts of predictors (e.g., predictions 1030).

In embodiments, a set of two modules, a reporting module 1012 and a visualization module 1034, may present results to a user and allow the user to control the system. As discussed herein, the visualization module 1034 may be a part of the CPP 1014 or it may be a separate entity, according to embodiments. The reporting module 1012 may create detailed individual-level reports 1032 that contain the predictions, cost impacts of individual predictors, and other features of interest. When ground truth costs are available in testing, the module may also output performance metrics including R², mean absolute percentage error, bias, and other measures based on ranking individuals by predicted cost. The variables and performance measures used in the reports 1032 can be configured. In embodiments, the visualization module 1034 may ingest outputs for interaction with the user through an interface 1036.

In embodiments, the system may include a user interface 1036 that configures and executes modules on-demand and independently of each other. The user interface may also report and record the status of the individual module executions (e.g., executions of modules 1002-1012 and 1034), which may help users decide on the next action. The outputs of previous experiments may be saved and reused by the downstream modules when desired. For example, configurations modularized into specific APIs may be generated for more accessible use, as described at FIGS. 6 and 7.

Various aspects of the modules described in the embodiment of the CPP architecture 1000 at FIG. 10 may be expanded upon further, for ease of understanding.

Data curation not present in the current art may be needed for use of the CPP 1014, as described herein. The first two modules in FIG. 10 may curate raw data from the user's database into a set of monthly sparse matrices that are ready for modeling. The two modules (e.g., data pull module 1002 and conversion module 1004), may create a layer of abstraction between the database and the building of models, which may greatly improve the efficiency of the CPP 1014. On one side, data extraction may involve many steps such as joining database tables, filtering out irrelevant data, cleaning the data, deriving additional features, and aggregating to the desired time resolution. On the other side, models may be created in the course of working with a single data source, corresponding to, for example, different input and output periods, feature sets, or algorithm settings. Without an intermediate data representation in between, the workflow could become quite burdensome.

The organization of the curated data into monthly units may also be convenient for both sides of the divide. Upstream, it may allow the database to be updated incrementally, for example, one month at a time, as new claims and enrollment records are generated. Since the system retains the sparse matrices from previous months, only the latest month's data has to be curated and added to the collection. Downstream, training and test datasets can be created “on demand” with complete flexibility in the input and output periods. This flexibility is seen in a variety of models presented herein.

The CPP 1014 of FIG. 10 may also provide for medical condition classification. The data pull module 1002 may allow for the derivation of any number of features to augment information in claims. Medical condition classification as an important example of derived features. In embodiments, medical condition classifications may be an important example of a derived feature, as they are a higher-level categorization of diagnosis codes provided on individual claim lines for justifying services. Condition classifications can be input to the model either in place of or in addition to the diagnosis codes.

In embodiments, two different classification systems are contemplated: Hierarchical Condition Categories (HCCs) and Disease Staging. HCCs are common risk adjustors for health cost prediction models covering a majority of chronic and acute conditions. Two different versions of HCC features have been implemented including Centers for Medicare and Medicaid Services (CMS-HCC) and Department of Health and Human Services (HHS-HCC). The CMS-HCC model has been adopted for the Medicare (65+ years) population while the HHS-HCC model has been implemented within commercial individual and small group markets. Construction of HCCs may be a two-step process in the 1014 pipeline. First, in the data pulling module 1002, individual diagnosis codes are mapped to condition categories (“CCs”) considering demographic data. HCCs are created during training testing data creation by considering all the CCs of patient and applying precedence rules.

Disease Staging by TRUVEN HEALTH ANALYTICS, an IBM company, (henceforth referred to as TRUVEN) is a clinically-based classification system that describes a patient's conditions as well as their severity level, both of which correlate with expected treatment and cost. Disease Staging is applied within the data pulling module to individual claim records. The output depends in general on all the diagnosis codes present in the claim along with secondary inputs such as age. One or more disease categories are produced along with accompanying stage numbers (e.g., 1.01, 2.03, etc.) indicating severity.

A system, for example, a CPP architecture 1000 as described at FIG. 10, may be configured to handle patients with varying histories. Healthcare cost prediction models like the one described herein may be trained on historical claims and enrollment data. However, individuals may often switch insurance or risk-sharing plans each year, or on a more frequent basis. Hence, individuals may end up with varying lengths of histories, from a few months to many years.

The system described herein may have flexibility to handle the problem of varying patient histories. One way is to define a single input period for all patients to ensure compatible features when training and applying the learners. This “one-size-fits-all” approach may fail to use all of the available data for individuals who began enrollment before the input period, whereas individuals who enrolled in the middle of the input period may either have incomplete data or have to be excluded entirely. A second way the system may handle the problem of varying patient histories includes a more adaptive alternative is to train multiple predictive learners, each corresponding to an input period of different length. Each patient may then be assigned to the input period that most closely matches their history, with variations on how this assignment is made.

In embodiments, regardless of how input periods are defined, to ensure that accurate and appropriate prediction models are created, individuals included in training and scoring may be required to satisfy coverage conditions during the input and output periods. In general, coverage conditions should strike a balance between maximizing the instances available for training and scoring while ensuring the data is statistically sound.

The following section may pertain to a predictive learning module, for example learning module 1008 of FIG. 10. The following may describe a particular embodiment, to provide a specific example of modeling and training of the pipeline.

In embodiments, the distribution of healthcare costs (the target variable for prediction) is both zero-inflated and heavy-tailed. In particular, there may be a few extremely high-cost individuals that can skew the predictive learner. This can present a problem not only in training but also in evaluations, where even a good model can appear significantly worse if it is “unlucky” in making large errors on outliers within a test set of limited size. As a simple remedy, both the training and evaluation modules in the pipeline have the ability to winsorize the right tail of the response variable, i.e., replace values above a threshold with the threshold.

The claims and eligibility data considered here typically have many sparse indicator variables that are potential predictors of the output cost. At the finest granularity, there can be tens of thousands of predictors in the form of diagnosis, procedure and drug codes. While some learning procedures (e.g., least absolute shrinkage and selection operator, or “LASSO”) may be able to perform feature selection during their training, others (e.g., random forests and other nonlinear models) could benefit from a pre-selection procedure that filters out variables with insignificant impact on the target variable. Even for procedures with feature selection capabilities, using pre-selection to eliminate obviously insignificant predictors can significantly reduce computational complexity.

The predictive learning module in the system pipeline meets this need by allowing any pre-selection algorithm to be run before the training data is passed to the main predictive learner. In addition to automatic feature pre-selection, the module is also able to force certain predictors to be included in or excluded from predictive learning regardless of their apparent relationship to the output. In this manner, domain knowledge about relevant and irrelevant predictors can be incorporated.

In general terms, the problem of learning to predict response Y from predictors X amounts to choosing an optimal predictor function \hat{ƒ} from a hypothesis class [symbol] to minimize the empirical risk,

$$\hat{f} = {argmin}_{f ∈ ℱ} \frac{1}{n} {\sum }_{i = 1}^{n} ℒ ( f ( {x}_{i} ) , {y}_{i} ) \qquad ( 1 )$$

where [symbol] is a loss function. In our datasets, the number of samples n and the number of predictors p can be large. Hence a rich-enough [symbol] is required; one that is robust in high-dimensional settings while providing accurate predictions.

Toward this end, the predictive learning module permits a multistage hypothesis class where the elements are the sum of elements from K different classes. This choice is flexible and can include several nonlinear models. Under the least square loss, the risk minimization in (1) can be written as:

$${\{ {\hat{f}}_{k} ∈ {ℱ}_{k} \}}_{k = 1}^{K} = {argmin}_{{\{ {f}_{k} ∈ {ℱ}_{k} \}}_{k = 1}^{K}} \frac{1}{n} {\sum }_{i = 1}^{n} {( {y}_{i} - {\sum }_{k = 1}^{K} {f}_{k} ( {x}_{i} ) )}^{2} \qquad ( 2 )$$

Where k indexes the component hypothesis class.

The module uses an alternating optimization approach to estimate {\hat{ƒ}ₖ∈ [symbol] ₖ}ₖ₌₁^{K} that works for any choice of hypothesis classes. All functions {\hat{ƒ}ₖ∈ [symbol] ₖ}ₖ₌₁^{K} are initially assumed to be identically zero and each one is then iteratively updated while fixing the others. The response variable to which the function ƒⱼ is fitted is given by

$${r}_{ij} = {y}_{i} - {\sum }_{{k = 1}_{k \neq j}}^{K} {\hat{f}}_{k} .$$

This may be iterated over estimating the K functions for several rounds until a stopping criteria is met.

In embodiments, in real-world deployments, the training dataset used to obtain \hat{ƒ} using (1) is usually historical while the testing data is current. The testing data's distribution could be different from that of the training data, for example due to geographic or health risk drifts. Assuming that the conditional distribution p_{Y|X} is the same between the two datasets, an optimal predictor function for the testing data can be obtained by re-weighting the training examples in (1) using the population shift weights wᵢ=q_{X}(xᵢ)/p_{X}(xᵢ), where p_{X} and q_{X} are the training and testing predictor distributions. The system described herein incorporates the capability for population shift correction using parametric or non-parametric approaches. The predictors used to compute the population shift weights can be different from those used in (2) for predictive modeling.

The system, for example CPP architecture 1000 of FIG. 10, may have a sensitivity analysis feature for assessing cost impacts, according to embodiments. In addition to cost predictions, users of the system may be interested in understanding the cost impacts of key predictors at the individual level, which can offer insights into the predictions. There are many ways of assessing the importance of individual features. For example, the cost impact of a continuous-valued feature [symbol] ⱼ on an instance i with feature values xᵢ can be quantified using the partial derivative ∂ƒ(x)/∂xⱼ evaluated at xᵢ. When ƒ is a linear function, this simply corresponds to the j^{th} coefficient and the cost impact is the same across all instances i. However, with nonlinear prediction functions, the cost impact may depend in general upon the instance-level features xᵢ.

The evaluation module in the predictive system allows a variety of sensitivity analysis methods for determining the cost impacts of individual predictors. For greater flexibility, the predictive learners should provide a common interface so that they can be called easily by the sensitivity analysis procedure, e.g., to produce new outputs from perturbed inputs. For efficiency, sensitivity analysis can be performed on one feature for all individuals simultaneously using vectorized sparse matrix operations, repeating for all desired features. In the end, the system reports the predictors with the highest cost impacts for each individual along with the cost impact values.

Sensitivity analysis can also be extended from individual predictors to a group of predictors. A more involved extension is to better handle predictors derived from other predictors, where varying one may have logical implications on others.

Besides understanding predicted costs and cost impacts of predictors at an individual level, it is important to provide similar insights at a cohort level, where cohorts can be defined by the user. For example, specific demographic subsets of cohorts can help an insurance company understand the health risks of the current enrolled population and possibly plan for future enrollment. The reporting module, for example reporting module 1012 of FIG. 10, may achieve this aim in some embodiments by aggregating individual predictions and cost impacts within each cohort defined by one or more variables. The aggregated cost impacts identify the key drivers of costs at the cohort level and describe the cohort health conditions.

The data used to develop and evaluate the system was obtained from the MARKETSCAN Research Database provided by TRUVEN. These databases contain over 240 million covered lives since 1995 sampled from members of large employers and health plans. The data provides insurance enrollment detail sand claims capturing diagnoses, procedures, pharmacy drug fills, associated costs, and other medical service utilizations. In embodiments, this may be the high-dimensional data referred to herein. The large sample size of the data allows for myriad, nationally representative studies involving cross-sectional and longitudinal views. This has made MARKETSCAN a highly published database in the United States with 500 studies in peer-reviewed journals in health policy, life sciences, and other health services research.

MARKETSCAN was used to create a unified data model containing eligibility and claim tables. The eligibility table provides enrollment and demographic information (e.g., age, geography, coverage type, etc.) at the per-member-per-month level. Months of continuous enrollment were calculated for each patient. The claims table includes detailed service line information for service within inpatient, outpatient, and outpatient pharmacy settings. For each claim line, this table captures the allowed and paid amounts and either diagnosis (coded using ICD-9-CM and ICD-10-CM systems) and medical procedures (CPT and ICD-9/ICD-10 procedures) for medical services, or pharmacy drug fills (drug therapeutic classes using TRUVEN REDBOOK).

For purposes of evaluation as reported in the following embodiments, cohorts of 1 million and 5 million patients spanning the years 2011-2015 were randomly selected from both the MARKETSCAN Commercial and Multi-State Medicaid databases. The data was further restricted to patients with both medical and pharmacy coverage and who are not on capitated health plans (e.g., health maintenance organizations). With capitated plans, a flat payment is typically made annually for each patient and thus claim-level cost data is either missing or heavily incomplete.

Specific examples of the reusability, configurability, and performance of the pipeline are highlighted herein, using two distinct applications. The first application illustrates on-demand partial-year prediction using the latest and richest possible data drawn from commercial insurance claims. The second is a “Cost of Care” application for a Medicaid population that estimates cost based primarily on a patient's health conditions. Both applications are realized through configuration of the system architecture and are evaluated using healthcare eligibility and claims data extracted from the MARKETSCAN databases.

In the first application, a cost prediction model for commercial claims may be developed using various sets of predictors and time periods. In particular, the system includes performance improvements with on-demand training using the latest available time periods as compared to a traditional model trained once on calendar-year periods. The value of using high-dimensional medical code features is also illustrated in the below example.

Data may be extracted from the unified MARKETSCAN Commercial Claims and Encounters database as described herein. Training data was constructed from years 2012 and 2013 and testing data from 2013 and 2014; the respective input and output periods are given later and do not overlap. The response variable for each individual is the average cost during the output period, computed only over enrolled months. In health insurance parlance, this cost may be referred to as the Per-Member-Per-Month (PMPM) cost. Feature vectors are formed form the following types of features taken over the input period: (a) demographic data including age, geographic location (coded at state level), and family size; (b) insurance plan including plan type (PPO, EPO, tec.) and months of continuous enrollment; (c) prior cost: total PMPM cost and PMPM costs for drug facility and professional claims in the input period. Division of the total cost into these three types is done by aggregating respective claim lines. In some cases, besides creating features that span the entire input period, temporal features may also be created by aggregating costs over the pre-specified intervals (e.g., quarters); and (d) medical codes including diagnosis, procedures, drug and HCC codes. HCC codes may be derived from diagnosis codes using the CMS-HCC mapping.

In embodiments, prior cost and medical code features may also be extracted using only claims serviced and paid within the input period. This restriction allows the model to be deployed immediately after the input period (hence “on-demand”) using the latest available data. In contrast, the output cost is obtained using claims serviced in the output period but paid up to 6 months after service, i.e., the runout period is 6 months.

Various embodiments may be used including benchmark (BM) method, prior-cost-only (PCO) method, and on-demand learning (ODL) strategies. The BM method may follow a traditional approach by training the predictive learner just once using features extracted from a calendar year 2012 and output cost from year 2013. Demographic and insurance plan features, HCCs and prior cost without any temporal subdivisions may be used. In a PCO method, the learner may be trained using the total prior cost from the full input period as its only predictor. The ODL method may use features extracted from the latest available data as specified herein. Two variations may be used, including a simple on-demand learner suing the same features as the BM method, and a rich on-demand learner which uses the BM features, as well as temporally subdivided costs and all medical codes as features.

The BM and ODL methods may use a 2-stage predictive learner with a LASSO learner in the first stage and a random forest (RF) learner in the second stage. The LASSO learner fits a linear function to the response and the RF learner fits the residual non-linear relationship; this ordering has been found to result in more interpretable cost impacts. The number of fitting rounds may be set to 2, and the PCO method uses a linear regressor with a single predictor. Features may be pre-selected for both LASSO and RF learners in the BM and ODL methods, specifically using their respective target responses before each is fit for the first time.

In embodiments, a univariate filter approach may be used, recognizing the limitations of ignoring features dependencies. Each indicator feature may be scored on the reduction using a simple model that partitions the training set by the value of the binary feature. Ranking features using this score is equivalent to ranking them based on a likelihood ratio test that assumes a Gaussian distribution for the output after outliers are trimmed from the extreme right tail. p-values corresponding to these feature scores may be estimated by simulating the null hypothesis of no effect using Monte Carlo experiments, each of which randomly permutes the target variable values in the training set. This direct approach to estimation of p-values has been adopted in various domains, since it does not depend on strong parametric assumptions. Features with p-values below an upper bound may then be selected.

Various training and testing protocols may be used, according to embodiments. For purposes of discussion, two different protocols are discussed herein. In a 12-month protocol, features are extracted from a 12-month input period going back from the latest month of data. In the early-months protocol, features are extracted from the same calendar year only. Note that in training, these protocols may apply only to the PCO and ODL methods; the BM method is trained only once as noted above. However, during the testing phase, the 12-month and early-months protocols apply to all methods.

To ensure sufficient quantity and quality of data, individuals included in the training and testing are required to have had at least 3 months of continuous enrollment spanning the input and output periods, and at least 1 month of enrollment in each of the input and output periods.

A PMPM cost in year 2014 (i.e., the testing output period) may be estimated by approximately 260,000 individuals who satisfy the coverage condition. In the first scenario, there is an assumption that there is data available until March 2014 to construct features. With the 12-month protocol, the testing input period is therefore April 2013 to March 2014 for all methods, and for the early-months protocol, the testing input period is the first three months of 2014. For the ODL and PCO methods, the training input period is set to be one year earlier, either April 2012 to March 2013 of the first three months of 2013. Note that the BM method, being static, may be trained once using features extracted from the 12 months of 2012. The four model varieties and the two different training/testing protocols result in 8 sets of results. The same experiment may be repeated for the second scenario where data is available until May 2014; early-months therefore mean 5 months. In this scenario, approximately 267,000 individuals satisfy the coverage condition. For populations of these size and all of the experiments reported here, the entire pipeline starting from pulling the data to reporting the results takes less than 3 hours of wall time to run utilizing 20 cores in a server running LINUX OS.

In embodiments, the percentage bias, R², and mean absolute percentage error (MAPE) of the predictions may be reported with testing data. MAPE is defined as the ratio between the mean absolute error (MAE) and the mean actual cost, expressed as a percentage. Furthermore, individuals may be ranked according to their predicted cost and choose the top 1% of the population. Using this top 1% cohort values may be computed including: (a) the percentage of this cohort who are also in the top 1% when ranked by the actual cost (top members at 1% measure); (b) the ratio of the total actual cost of this cohort compared to the true top 1% of the population, also expressed as a percentage (Top Cost at 1% measure). The last two measures may be important in identifying the riskiest population (in terms of cost) for further analysis. The performance of the predictive learners with input data as of March 2014 is reported in Table 1 (below). The BM and simple ODL methods are directly comparable since the only difference is that simple ODL uses on-demand training with April 2012-March 2013 input data. The simple ODL method has better performance in all measures because of this on-demand training. Improved performance in all measure except % Bias is achieved by the rich ODL learner, with the bias within 1 percentage point of the best. Thus, including a richer feature set (temporally subdivided prior cost and medical codes) further improves the performance. A subtle point to note is that compared to the simple ODL method, the rich ODL method adds test of thousands of medical code features increasing the data dimensionality tremendously. However, the feature pre-selection approach described herein as well as the high capacity 2-stage model is able to efficiently handle the high dimensionality and extract useful information from the additional features.

It is also instructive to compare the early 3-month and 12-month protocols. For the simple and rich ODL methods, using 12 months of historical data provides improved performance for the latter but not for the former (except for bias). This may demonstrate that when prior costs are temporally subdivided and all medical code features are included, the rich ODL model benefits from aggregation over a longer temporal range. In contrast, for the simple ODL model, the predictions are mainly driven by prior costs aggregated over the entire input period, and not considering the previous year in this aggregation is better.

In the 12-month protocol, it is possible that a good fraction of individuals do not have continuous enrollment over the entire 12 months of the input period. There may be at least three possible ways to handle this situation. In a simple ODL method, features are created based on the entire input period without any finer temporal aggregation. In contrast, the rich ODL method assumes zero cost values during months in which there is no enrollment when aggregating the prior cost over intervals within the input period. A third approach as described herein, is to create multiple learners for patients with different histories.

Table 1 (below) also includes the performance of the learning methods with input data as of May 2014. Having more months of data from the prediction year improves all performance measures. The comparisons made previously between the BM, simple and rich ODL methods hold generally except for one difference. For the rich ODL method, the early 5-month protocol results in better R2 than the 12-month protocol while other measures are close. A likely reason is that more historical data from the current year is available, less relevant data from the previous year starts to hurt the predictions.

Finally, with the simple PCO method, the R², Top members, and Top cost measures are close to but lower than those of the simple ODL method. The general trend in performance with the on-demand learners is in line with the feature sets that they use: rich ODL is better than simple ODL which is better than PCO. Thus, prior costs can provide a good baseline prediction but adding more features and considering finer temporal aggregations of prior cost can provide substantial boosts.

In embodiments, Table 1 (below), may be titled “Performance of Cost Prediction Models Using Data Until March/May 2014”

TABLE 1

| | | March 2014 | | | | | May 2014 | | | | |
|---|---|---|---|---|---|---|---|---|---|---|---|
| | | | | | Top | Top | | | | Top | Top |
| | Model | Bias | R² | | members | cost | Bias | R² | | members | cost |
| Prot. | Version | (%) | (%) | MAPE | at 1% | at 1% | (%) | (%) | MAPE | at 1% | at 1% |
| early- | BM | −13.36 | 28.88 | 80.11 | 41.50 | 61.97 | −6.65 | 38.68 | 75.53 | 50.67 | 71.86 |
| 3 m/5 m | ODL/simp. | −3.42 | 34.74 | 79.79 | 43.59 | 63.87 | −3.22 | 52.54 | 63.73 | 58.45 | 76.88 |
| | ODL/rich | −3.94 | 37.03 | 77.59 | 45.88 | 65.45 | −2.85 | 53.92 | 62.75 | 58.98 | 78.19 |
| | PCO | −3.68 | 31.02 | 87.26 | 41.26 | 60.84 | −3.30 | 50.42 | 68.22 | 56.26 | 74.77 |
| 12 m | BM | −3.02 | 26.66 | 88.95 | 38.70 | 58.22 | −1.32 | 33.55 | 84.27 | 45.23 | 65.70 |
| | ODL/simp. | −2.76 | 28.70 | 85.88 | 41.20 | 60.41 | −2.92 | 40.89 | 73.68 | 51.74 | 70.07 |
| | ODL/rich | −3.04 | 40.11 | 75.34 | 48.02 | 67.59 | −2.68 | 51.05 | 63.11 | 59.49 | 77.50 |
| | PCO | −3.61 | 25.41 | 93.66 | 39.66 | 57.75 | −3.20 | 39.03 | 78.55 | 51.63 | 68.93 |

In embodiments, a Cost of Care (CoC) model has been developed to estimate the cost based primarily on a patient's medical conditions as well as demographic and insurance plan characteristics. The CoC model has a number of differences with respect to the commercial claims model described herein, as well as many model varieties, all of which can be accommodated through configuration of the system.

There may be differences between the CoC model described for the system herein and a commercial claims model. First, for example, the CoC model does not consider treatments (e.g., procedures and drugs) that the patient has received or the prior cost of such treatments. Instead, the intent is to estimate an average, “standard” cost for a given medical risk profile, which can then be used to adjust actual costs of patients with the same profile. Such risk adjustment may be useful for example, in fairly assessing and comparing the cost effectiveness of healthcare providers. Exclusion of prior cost and treatment variables from the model is easily done during training and test data creation. The CoC model may accordingly be less accurate than the commercial claims prediction model at the individual level. Second, the CoC model uses the Disease Staging classification described herein, instead of the HCC codes. Third, the input period may be defined differently. Instead of considering only claims that are serviced and paid within the input period as in the commercial claims model, the CoC model considers claims serviced within the input period and paid within a 6-month runout period after the service date. The advantage is that more claims are included and model accuracy increases relative to the “on-demand” option, as described herein. A disadvantage is that users may be required to wait for the runout period to pass and the most recent (albeit incomplete) data is not used. Either option can be implemented in the training and test data creation module in the present system by changing the range of monthly data that is aggregated. These models are managed in a similar way to the multiple models for different patient histories discussed herein.

In embodiments, varieties of the CoC model may be used, by varying dimensions through the configuration of the system. One variety includes a prospective model using inputs from a 12-month period to predict cost for the subsequent 12-month period. The concurrent model estimates cost using inputs from the same 12-month period (e.g., for retrospective risk adjustment). The difference lies in the input period. A second variation may use data from partial year or full year enrollees (e.g., members who have been enrolled for the specified amount of time). The models may accept and make predictions for patients with as little as one month of enrollment within the single 12-month period for concurrent models, and within each of the 12-month input and prediction periods for prospective models. The models may also be restricted (both in training and testing) to patients with 12- or 24-month continuous enrollment respectively. This is controlled by the coverage condition parameters in the training and test data creation module. A third variation may allow for the inclusion of medical and drug cost considerations versus medical-only considerations. The target variable can be configured to be either a patient's total cost for medical services and prescription drugs or the cost for medical services only. Finally, cost censoring levels may vary in the implementation of various embodiments. For example, in particular embodiments, the default may be to winsorize at the 99.99 percentile (1 in 10⁴) to prevent a few outliers from distorting evaluation results, or at $250,000 to compare with other published results.

Similar to the commercial claims model, the CoC model may also use a 2-stage predictive learner (LASSO+RF) with two fitting rounds, as well as feature preselection.

In embodiments, data analysis may be conducted as follows. In embodiments, the MARKETSCAN MULTI-STATE MEDICAID DATABASE may be used to develop and evaluate the CoC models, specifically data from years 2011-2014. In addition to the filters described herein, patients who were eligible for both Medicaid and Medicare were excluded since their payment data was incomplete. Variables used in the model may include the following: medical data, demographic data, and insurance plan data. For example, medical data could include disease staging features and diagnosis codes. The default is to use Disease staging features only but embodiments may also include the use of both disease staging and diagnosis codes. Demographic data may include, for example, age, basis of Medicaid eligibility, and federal maintenance assistance status. The latter two may indicate why a patient qualifies for Medicaid and can be predictive of cost. Also of note, while data comes from health plans and governments in multiple US states, no state-specific or indeed any geographic information is available due to the need to anonymize the data sources. Thus, the models cannot capture variations between state Medicaid programs.

Finally, embodiments may vary in their results. In particular embodiments, all models were trained on one time period and tested on the corresponding period one year later. Thus given the 4 years of MARKETSCAN data, prospective models were trained on the periods 2011-2012 and 2012-2013 (input year-prediction year) and tested on 2012-2013 and 2013-2014, respectively. Concurrent models were trained on 2011, 2012, 2013, and tested on 2012, 2013, 2014, respectively.

Table 2 (below) shows the individual-level accuracy metrics achieved by the prospective medical and drug cost models, both R2 and MAPE. The default model uses the following options discussed previously: disease staging features without diagnosis codes, including partial-year enrollees with as little as one month of enrollment, and cost winsorizing at the 99.99 percentile. The other models differ from the default only in the aspect indicated in Table 2 (below). All results depicted were obtained from a 1 million patient sample except for the ‘5× sample”, which is from a 5 million sample. The latter shows the significant benefit of training the same model on a larger sample. The third row demonstrates that retaining diagnosis codes alongside disease staging also improves accuracy by a smaller amount. The last two rows represent easier situations where accuracy is expected to be much higher: restriction to full-year enrollees with complete data, and winsorizing at $250,000, thereby mitigating high-cost outliers.

Table 2 (below) shows accuracy results for concurrent medical-only cost models. Concurrent models are naturally much more accurate because the input and prediction periods are the same, i.e., there is no need to predict the future. Otherwise, the pattern is similar to Table 2 (below) but the magnitude of the differences is larger.

Table 2, below, is titled “Accuracy of Prospective and Concurrent Cost Models”:

| | Prospective Medical + | | | | | | | | | | | | | | |
|---|---|---|---|---|---|---|---|---|---|---|---|---|---|---|---|
| | Drug Cost Models | | | | | | Concurrent Medical-Only Cost Models | | | | | | | | |
| | 2012-2013 | | | 2013-2014 | | | 2012 | | | 2013 | | | 2014 | | |
| Model | | R² | | | R² | | | R² | | | R² | | | R² | |
| Version | n | (%) | MAPE | n | (%) | MAPE | n | (%) | MAPE | n | (%) | MAPE | n | (%) | MAPE |
| Default | 145k | 24.5 | 88.1 | 151k | 25.8 | 85.7 | 204k | 42.6 | 72.0 | 207k | 39.3 | 74.5 | 214k | 35.7 | 77.8 |
| 5X sample | 740k | 27.5 | 81.4 | 756k | 28.9 | 83.2 | 1019k | 48.1 | 66.6 | 1034k | 46.8 | 70.3 | 1070k | 41.7 | 73.9 |
| Include Dx codes | 148k | 25.6 | 85.5 | 151k | 26.1 | 85.4 | 204k | 44.3 | 70.6 | 207k | 41.7 | 73.9 | 214k | 37.4 | 77.3 |
| Full-year only | 89k | 35.2 | 80.0 | 100k | 36.0 | 74.7 | 115k | 57.9 | 59.8 | 121k | 58.0 | 63.0 | 133k | 54.9 | 63.9 |
| $250K winsorizing | 148k | 35.8 | 84.7 | 151k | 40.9 | 81.9 | 204k | 63.6 | 59.1 | 207k | 61.3 | 62.7 | 21.4k | 58.3 | 64.4 |

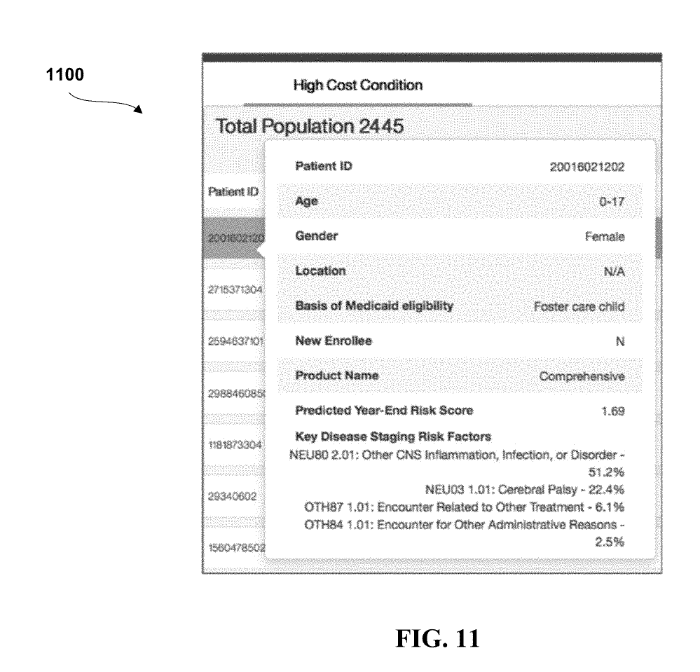

FIG. 11 may depict a sample UI 1100 of an individual report, according to embodiments. The sample individual report may be from the visualization module (e.g., visualizer 1034 of FIG. 10), that displays the prediction for an individual (normalized to a risk score), key risk factors that affect the score, and other information. The sensitivity analysis method used to generate the risk factors focuses on disease staging features. For each disease category (e.g., ‘NEU80’), there is one feature at the category level as well as multiple features corresponding to different stages (e.g., 1.01, 2.01) of the disease category. These are all treated as one group. An approach of “turning off” one disease category at a time, i.e., setting all features in the group to zero while leaving other features unchanged, may be used and re-computing the predicted cost. This method can be seen as a variation upon individual condition expectation. The cost impact of a disease category is then the difference between the nominal prediction and the prediction obtained after turning it off, expressed as a percentage of the nominal prediction. The reported risk factors inform users of medical conditions that lead to the elevated risk score.

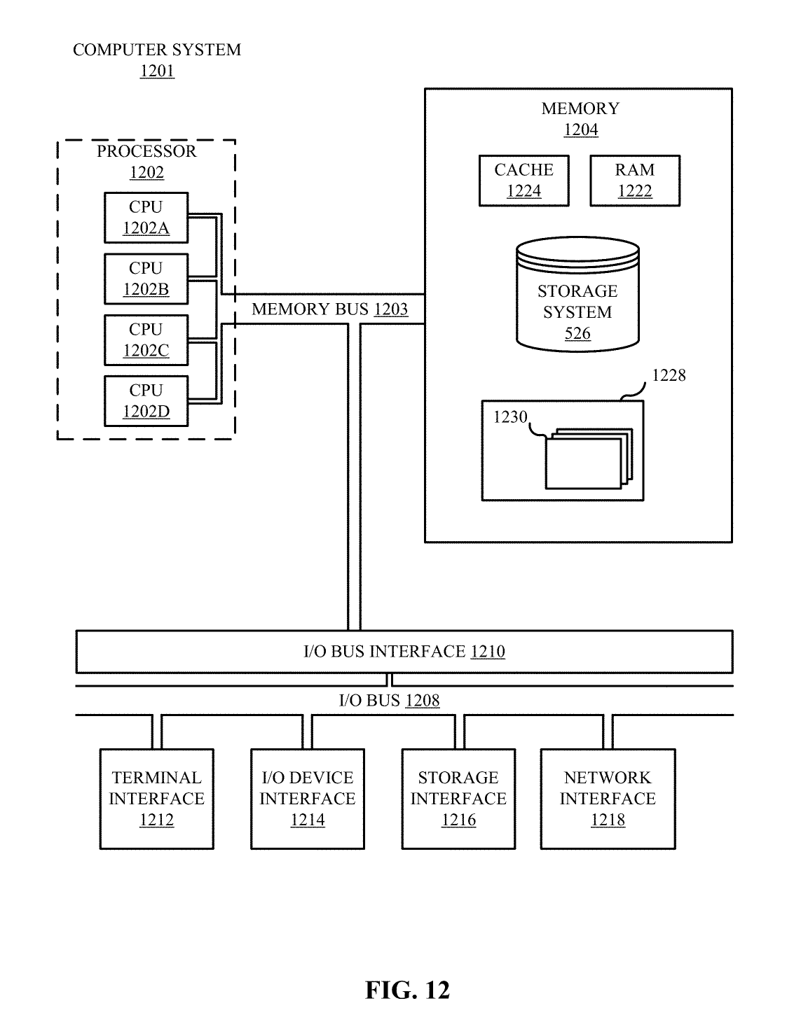

Referring now to FIG. 12, shown is a high-level block diagram of an example computer system 1201 (e.g., devices 105, server 135, device 305, device 350, ontology system 301) that can be used in implementing one or more of the methods, tools, and modules, and any related functions, described herein (e.g., using one or more processor circuits or computer processors of the computer), in accordance with embodiments of the present disclosure. In some embodiments, the major components of the computer system 1201 can comprise one or more CPUs 1202, a memory subsystem 1204, a terminal interface 1212, a storage interface 1216, an I/O (Input/Output) device interface 1214, and a network interface 1218, all of which can be communicatively coupled, directly or indirectly, for inter-component communication via a memory bus 1203, an I/O bus 1208, and an I/O bus interface unit 1210.

The computer system 1201 can contain one or more general-purpose programmable central processing units (CPUs) 1202A, 1202B, 1202C, and 1202D, herein generically referred to as the CPU 1202. In some embodiments, the computer system 1201 can contain multiple processors typical of a relatively large system; however, in other embodiments the computer system 1201 can alternatively be a single CPU system. Each CPU 1202 can execute instructions stored in the memory subsystem 1204 and can include one or more levels of on-board cache.

System memory 1204 can include computer system readable media in the form of volatile memory, such as random access memory (RAM) 1222 or cache memory 1224. Computer system 1201 can further include other removable/non-removable, volatile/non-volatile computer system storage media. By way of example only, storage system 1226 can be provided for reading from and writing to a non-removable, non-volatile magnetic media, such as a “hard-drive.” Although not shown, a magnetic disk drive for reading from and writing to a removable, non-volatile magnetic disk (e.g., a “floppy disk”), or an optical disk drive for reading from or writing to a removable, non-volatile optical disc such as a CD-ROM, DVD-ROM or other optical media can be provided. In addition, memory 1204 can include flash memory, e.g., a flash memory stick drive or a flash drive. Memory devices can be connected to memory bus 1203 by one or more data media interfaces. The memory 1204 can include at least one program product having a set (e.g., at least one) of program modules that are configured to carry out the functions of various embodiments.

One or more programs/utilities 1228, each having at least one set of program modules 1230 can be stored in memory 1204. The programs/utilities 1228 can include a hypervisor (also referred to as a virtual machine monitor), one or more operating systems, one or more application programs, other program modules, and program data. Each of the operating systems, one or more application programs, other program modules, and program data or some combination thereof, can include an implementation of a networking environment. Programs 1228 and/or program modules 1230 generally perform the functions or methodologies of various embodiments.

In some embodiments, the program modules 1230 of the computer system 1201 can include various modules of the pipeline, as described herein.

Although the memory bus 1203 is shown in FIG. 12 as a single bus structure providing a direct communication path among the CPUs 1202, the memory subsystem 1204, and the I/O bus interface 1210, the memory bus 1203 can, in some embodiments, include multiple different buses or communication paths, which can be arranged in any of various forms, such as point-to-point links in hierarchical, star or web configurations, multiple hierarchical buses, parallel and redundant paths, or any other appropriate type of configuration. Furthermore, while the I/O bus interface 1210 and the I/O bus 1208 are shown as single respective units, the computer system 1201 can, in some embodiments, contain multiple I/O bus interface units 1210, multiple I/O buses 1208, or both. Further, while multiple I/O interface units are shown, which separate the I/O bus 1208 from various communications paths running to the various I/O devices, in other embodiments some or all of the I/O devices can be connected directly to one or more system I/O buses.

In some embodiments, the computer system 1201 can be a multi-user mainframe computer system, a single-user system, or a server computer or similar device that has little or no direct user interface, but receives requests from other computer systems (clients). Further, in some embodiments, the computer system 1201 can be implemented as a desktop computer, portable computer, laptop or notebook computer, tablet computer, pocket computer, telephone, smart phone, network switches or routers, or any other appropriate type of electronic device.

It is noted that FIG. 12 is intended to depict the representative major components of an exemplary computer system 1201. In some embodiments, however, individual components can have greater or lesser complexity than as represented in FIG. 12, components other than or in addition to those shown in FIG. 12 can be present, and the number, type, and configuration of such components can vary.

As discussed in more detail herein, it is contemplated that some or all of the operations of some of the embodiments of methods described herein can be performed in alternative orders or may not be performed at all; furthermore, multiple operations can occur at the same time or as an internal part of a larger process.

It is to be understood that although this disclosure includes a detailed description on cloud computing, implementation of the teachings recited herein are not limited to a cloud computing environment. Rather, embodiments of the present invention are capable of being implemented in conjunction with any other type of computing environment now known or later developed.

Cloud computing is a model of service delivery for enabling convenient, on-demand network access to a shared pool of configurable computing resources (e.g., networks, network bandwidth, servers, processing, memory, storage, applications, virtual machines, and services) that can be rapidly provisioned and released with minimal management effort or interaction with a provider of the service. This cloud model may include at least five characteristics, at least three service models, and at least four deployment models.

Characteristics are as follows:

On-demand self-service: a cloud consumer can unilaterally provision computing capabilities, such as server time and network storage, as needed automatically without requiring human interaction with the service's provider.

Broad network access: capabilities are available over a network and accessed through standard mechanisms that promote use by heterogeneous thin or thick client platforms (e.g., mobile phones, laptops, and PDAs).

Resource pooling: the provider's computing resources are pooled to serve multiple consumers using a multi-tenant model, with different physical and virtual resources dynamically assigned and reassigned according to demand. There is a sense of location independence in that the consumer generally has no control or knowledge over the exact location of the provided resources but may be able to specify location at a higher level of abstraction (e.g., country, state, or datacenter).

Rapid elasticity: capabilities can be rapidly and elastically provisioned, in some cases automatically, to quickly scale out and rapidly released to quickly scale in. To the consumer, the capabilities available for provisioning often appear to be unlimited and can be purchased in any quantity at any time.

Measured service: cloud systems automatically control and optimize resource use by leveraging a metering capability at some level of abstraction appropriate to the type of service (e.g., storage, processing, bandwidth, and active user accounts). Resource usage can be monitored, controlled, and reported, providing transparency for both the provider and consumer of the utilized service.

Service Models are as follows:

Software as a Service (SaaS): the capability provided to the consumer is to use the provider's applications running on a cloud infrastructure. The applications are accessible from various client devices through a thin client interface such as a web browser (e.g., web-based e-mail). The consumer does not manage or control the underlying cloud infrastructure including network, servers, operating systems, storage, or even individual application capabilities, with the possible exception of limited user-specific application configuration settings.

Platform as a Service (PaaS): the capability provided to the consumer is to deploy onto the cloud infrastructure consumer-created or acquired applications created using programming languages and tools supported by the provider. The consumer does not manage or control the underlying cloud infrastructure including networks, servers, operating systems, or storage, but has control over the deployed applications and possibly application hosting environment configurations.

Infrastructure as a Service (IaaS): the capability provided to the consumer is to provision processing, storage, networks, and other fundamental computing resources where the consumer is able to deploy and run arbitrary software, which can include operating systems and applications. The consumer does not manage or control the underlying cloud infrastructure but has control over operating systems, storage, deployed applications, and possibly limited control of select networking components (e.g., host firewalls).

Deployment Models are as follows:

Private cloud: the cloud infrastructure is operated solely for an organization. It may be managed by the organization or a third party and may exist on-premises or off-premises.

Community cloud: the cloud infrastructure is shared by several organizations and supports a specific community that has shared concerns (e.g., mission, security requirements, policy, and compliance considerations). It may be managed by the organizations or a third party and may exist on-premises or off-premises.

Public cloud: the cloud infrastructure is made available to the general public or a large industry group and is owned by an organization selling cloud services.

Hybrid cloud: the cloud infrastructure is a composition of two or more clouds (private, community, or public) that remain unique entities but are bound together by standardized or proprietary technology that enables data and application portability (e.g., cloud bursting for load-balancing between clouds).

A cloud computing environment is service oriented with a focus on statelessness, low coupling, modularity, and semantic interoperability. At the heart of cloud computing is an infrastructure that includes a network of interconnected nodes.

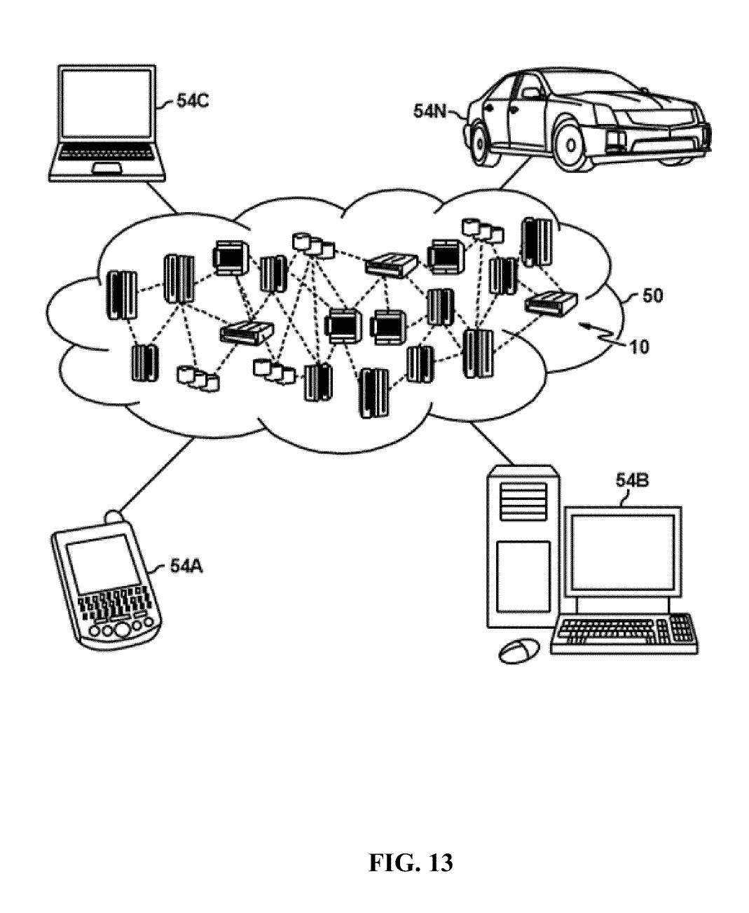

Referring now to FIG. 13, illustrative cloud computing environment 50 is depicted. As shown, cloud computing environment 50 includes one or more cloud computing nodes 10 with which local computing devices used by cloud consumers, such as, for example, personal digital assistant (PDA) or cellular telephone 54A, desktop computer 54B, laptop computer 54C, and/or automobile computer system 54N may communicate. Nodes 10 may communicate with one another. They may be grouped (not shown) physically or virtually, in one or more networks, such as Private, Community, Public, or Hybrid clouds as described hereinabove, or a combination thereof. This allows cloud computing environment 50 to offer infrastructure, platforms and/or software as services for which a cloud consumer does not need to maintain resources on a local computing device. It is understood that the types of computing devices 54A-N shown in FIG. 13 are intended to be illustrative only and that computing nodes 10 and cloud computing environment 50 can communicate with any type of computerized device over any type of network and/or network addressable connection (e.g., using a web browser).

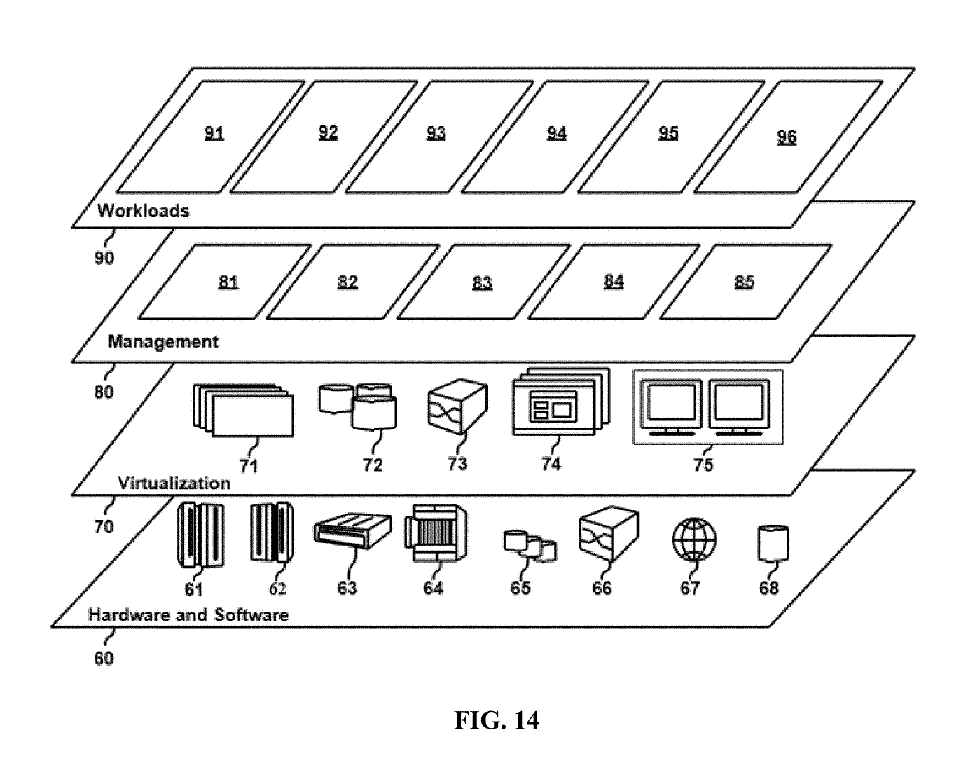

Referring now to FIG. 14, a set of functional abstraction layers provided by cloud computing environment 50 (FIG. 13) is shown. It should be understood in advance that the components, layers, and functions shown in FIG. 14 are intended to be illustrative only and embodiments of the invention are not limited thereto. As depicted, the following layers and corresponding functions are provided:

Hardware and software layer 60 includes hardware and software components. Examples of hardware components include: mainframes 61; RISC (Reduced Instruction Set Computer) architecture based servers 62; servers 63; blade servers 64; storage devices 65; and networks and networking components 66. In some embodiments, software components include network application server software 67 and database software 68.

Virtualization layer 70 provides an abstraction layer from which the following examples of virtual entities may be provided: virtual servers 71; virtual storage 72; virtual networks 73, including virtual private networks; virtual applications and operating systems 74; and virtual clients 75.

In one example, management layer 80 may provide the functions described below. Resource provisioning 81 provides dynamic procurement of computing resources and other resources that are utilized to perform tasks within the cloud computing environment. Metering and Pricing 82 provide cost tracking as resources are utilized within the cloud computing environment, and billing or invoicing for consumption of these resources. In one example, these resources may include application software licenses. Security provides identity verification for cloud consumers and tasks, as well as protection for data and other resources. User portal 83 provides access to the cloud computing environment for consumers and system administrators. Service level management 84 provides cloud computing resource allocation and management such that required service levels are met. Service Level Agreement (SLA) planning and fulfillment 85 provide pre-arrangement for, and procurement of, cloud computing resources for which a future requirement is anticipated in accordance with an SLA.

Workloads layer 90 provides examples of functionality for which the cloud computing environment may be utilized. Examples of workloads and functions which may be provided from this layer include: mapping and navigation 91; software development and lifecycle management 92; virtual classroom education delivery 93; data analytics processing 94; transaction processing 95; and healthcare cost prediction 96.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to explain the principles of the embodiments, the practical application or technical improvement over technologies found in the marketplace, or to enable others of ordinary skill in the art to understand the embodiments disclosed herein.

What is claimed is:

1. A computer-implemented method for generating predictive data, the method comprising:
building, based on a set of historical training data for a set of training members, a trained configurable predictive pipeline;
identifying, based on empirical testing of the set of historical training data, a set of member-level features;
generating, using the trained configurable predictive pipeline and a set of historical test data for the set of testing members, a set of predictive data for each member in the set of testing members; and
generating, for the set of predictive data for each member, a predictive report, wherein the predictive report comprises the set of predictive data and a set of explanations, wherein the predictive report is customizable for various levels of granularity including cohort levels, and wherein the set of explanations provides details for each prediction in the report.

2. The computer-implemented method of claim 1, wherein the building comprises:
training, using a first subset of the set of historical training data, the configurable predictive pipeline;
predicting, using the configurable predictive pipeline, a predictive second subset of the set of historical training data;
comparing, with a second subset of the set of historical training data, the predictive second subset of the set of historical training data; and
modifying, based on the comparing, weighting in the configurable predictive pipeline.

3. The computer-implemented method of claim 1, wherein the method further comprises:
curating, prior to the building, the set of historical training data for the set of training members and the set of historical test data for the set of testing members.

4. The computer-implemented method of claim 3, wherein the curating comprises data standardization and the use of sparse matrices, and wherein the data curation addresses temporal drifts in population.

5. The computer-implemented method of claim 1, wherein the details comprise data from a treatment course

6. The computer-implemented method of claim 1, wherein the method further comprises: saving, to memory and responsive to the building the trained configurable predictive pipeline, a set of building configurations; and saving, responsive to the generating the set of predictive data for each member, a set of generating configurations, wherein each set of configurations is accessible in a modularized format.

7. The computer-implemented method of claim 1, wherein the building further comprises:
identifying, prior to the building and for the set of training members, a first subset of members and a second subset of members, wherein the first subset of members is identified based on a first duration of membership and the second subset of members is identified based on a second duration of membership;
training, for the first subset of members, a first subset model; and
training, for the second subset of members, a second subset model;
and wherein the generating the set of predictive data for each member further comprises:
generating, using the first subset model, a first subset of predictive data;
generating, using the second subset model, a second subset of predictive data; and
merging the first subset of predictive data and the second subset of predictive data into the set of predictive data for each member.

8. The computer-implemented method of claim 1, wherein the set of testing members is a subset of the set of training members.

9. A system comprising:
a training module configured to build, based on a set of historical training data for a set of training members, a trained configurable predictive pipeline;
a feature identification module configured to identify, based on empirical testing of the set of historical training data, a set of member-level features;
a prediction module configured to generate, using the trained configurable predictive pipeline and a set of historical test data for the set of testing members, a set of predictive data for each member in the set of testing members, wherein the predictive data comprise predictions for each of the set of member-level features; and
a reporting module configured to:
generate, for the set of predictive data for each member in the set of testing members, a predictive report, wherein the predictive report comprises the set of predictive data and a set of explanations, and wherein the set of explanations provides details for each prediction in the report.

10. The system of claim 9, wherein the training module is further configured to build the trained configurable predictive pipeline by:
training, using a first subset of the set of historical training data, the configurable predictive pipeline;
predicting, using the configurable predictive pipeline, a predictive second subset of the set of historical training data;
comparing, with a second subset of the set of historical training data, the predictive second subset of the set of historical training data; and
modifying, based on the comparing, weighting in the configurable predictive pipeline.

11. The system of claim 9, wherein the training module is further configured to:
curate, before the building, the set of historical training data for the set of training members and the set of historical test data for the set of testing members.

12. The system of claim 11, wherein the curating comprises data standardization and the use of sparse matrices.

13. The system of claim 9, wherein the details comprise data from a treatment course.

14. The system of claim 9, wherein the predictive report is configurable to varying levels of granularity including member level and cohort level reporting.

15. The system of claim 9, wherein the building module is further configured to:
identify, prior to the building and for the set of training members, a first subset of members and a second subset of members, wherein the first subset of members is identified based on a first duration of membership and the second subset of members is identified based on a second duration of membership;
train, for the first subset of members, a first subset model; and
train, for the second subset of members, a second subset model;
and wherein the prediction module is further configured to generate the set of predictive data for each member in the set of testing members by:
generating, using the first subset model, a first subset of predictive data;
generating, using the second subset model, a second subset of predictive data; and
merging the first subset of predictive data and the second subset of predictive data into the set of predictive data for each member.

16. The system of claim 9, wherein the set of testing members is a subset of the set of training members.

17. A computer program product comprising a computer readable storage medium having program instructions embodied therewith, wherein the computer readable storage medium is not a transitory signal per se, the program instructions executable by a processor to cause the processor to perform a method comprising:
building, based on a set of historical training data for a set of training members, a trained configurable predictive pipeline;
identifying, based on empirical testing of the set of historical training data, a set of member-level features;
generating, using the trained configurable predictive pipeline and a set of historical test data for the set of testing members, a set of predictive data for each member in the set of testing members; and
generating, for the set of predictive data for each member, a predictive report, wherein the predictive report comprises the set of predictive data and a set of explanations, and wherein the set of explanations provides details for each prediction in the report.

18. The computer program product of claim 17, wherein the building further comprises:
training, using a first subset of the set of historical training data, the configurable predictive pipeline;
predicting, using the configurable predictive pipeline, a predictive second subset of the set of historical training data;
comparing, with a second subset of the set of historical training data, the predictive second subset of the set of historical training data; and
modifying, based on the comparing, weighting in the configurable predictive pipeline.

19. The computer program product of claim 17, wherein the building further comprises:
identifying, prior to the building and for the set of training members, a first subset of members and a second subset of members, wherein the first subset of members is identified based on a first duration of membership and the second subset of members is identified based on a second duration of membership;
training, for the first subset of members, a first subset model; and
training, for the second subset of members, a second subset model;
and wherein the generating the set of predictive data for each member further comprises:
generating, using the first subset model, a first subset of predictive data;
generating, using the second subset model, a second subset of predictive data; and
merging the first subset of predictive data and the second subset of predictive data into the set of predictive data for each member.

20. The computer program product of claim 17, wherein the details comprise data from a treatment course.

21. A computer-implemented method for generating predictive healthcare cost data, the method comprising:
identifying, based on empirical testing of a set of historical patient training data for a set of training members, a set of member-level features;
generating, using a trained configurable predictive healthcare cost pipeline and a set of historical patient test data for a set of testing members in a testing set, a set of predictive data for each member in the set of testing members; and
generating, for the set of predictive data for each member, a predictive report, wherein the predictive report comprises a set of medical risk factors, a predictive risk score, and a set of predictive costs.

22. The computer-implemented method of claim 21, wherein the method further comprises: building, prior to the identifying and based on the set of historical patient training data for the set of training members, the trained configurable predictive healthcare cost pipeline, wherein the set of training members are a set of members enrolled in a particular health insurance program and wherein the set of historical patient training data and the set of historical patient testing data comprise high-dimensional data.

23. The computer-implemented method of claim 22, wherein the high-dimensional data comprises patient demographic data, treatment course data, and diagnosis data.

24. A system for generating predictive healthcare cost data comprising:
a computer readable storage medium with program instructions stored thereon; and
one or more processors configured to execute the program instructions to perform a method comprising:
building, based on a set of historical patient training data for a set of training members, a trained configurable predictive healthcare cost pipeline, wherein the set of training members are a set of members enrolled in a particular health insurance program;
identifying, based on empirical testing of the set of historical patient training data for the set of training members, a set of member-level features;
generating, using the trained configurable predictive healthcare cost pipeline and a set of historical patient test data for a set of testing members in a testing set, a set of predictive data for each member in the set of testing members; and
generating, for the set of predictive data for each member, a predictive report, wherein the predictive report comprises at set of medical risk factors, a predictive risk score, and a set of predictive costs.

25. The system of claim 24, wherein the method further comprises curating, prior to the building, the set of historical patient training data for the set of training members and the set of historical patient test data for the set of testing members.
